SEQUENCE LISTING (4 sequences)

SEQ ID NO: 1 - protein, 220 aa, Homo sapiens
DVVMTQTPLSLPVTPGEPASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 442 aa, Homo sapiens
EVQLVQSGAEVEKPGASVKISCKASGSSFTGYNMNWVRQNIGKSLEWIGAIDPYYGGTSYNQKFKGRATLTVDKSTSTAYMHLKSLRSEDTAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 441 aa, Homo sapiens
EVQLVQSGAEVEKPGASVKISCKASGSSFTGYNMNWVRQNIGKSLEWIGAIDPYYGGTSYNQKFKGRATLTVDKSTSTAYMHLKSLRSEDTAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 441 aa, Homo sapiens
EVQLVQSGAEVEKPGASVKISCKASGSSFTGYNMNWVRQNIGKSLEWIGAIDPYYGGTSYNQKFKGRATLTVDKSTSTAYMHLKSLRSEDTAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2025/0186603 A1
(43) Pub. Date: Jun. 12, 2025

(54) ANTI-GD2 ANTIBODIES, IMMUNOCONJUGATES AND THERAPEUTIC USES THEREOF

(71) Applicant: Merck Patent GmbH, Darmstadt (DE)

(72) Inventors: Sean D. MCKENNA, Duxbury, MA (US); Alec GROSS, Newton, MA (US); Jianguo MA, Newton, MA (US); Julia DOTTERWEICH, Darmstadt (DE); Nicolas RASCHE, Darmstadt (DE); Min SHAN, Darmstadt (DE); Christiane AMENDT, Muehltal (DE); Jan ANDERL, Lautertal (DE); Willem SLOOT, Heesch (NL)

(21) Appl. No.: 18/844,775

(22) PCT Filed: Mar. 8, 2023
(86) PCT No.: PCT/US2023/063957

§ 371 Date: Sep. 6, 2024

Related U.S. Application Data
(60) Provisional application No. 63/488,269, filed on Mar. 3, 2023.
(60) Provisional application No. 63/318,256, filed on Mar. 9, 2022.

(51) Int. Cl. A61K 47/68; A61P 35/00; C07K 16/30

(52) U.S. Cl. CPC A61K 47/68037; A61K 47/6865; A61K 47/6889; A61P 35/00; C07K 16/3084

(57) ABSTRACT

The invention provides antibodies which bind human GD2 protein, as well as isolated nucleic acids and host cells comprising a sequence encoding said antibodies. The invention also provides immunoconjugates comprising said antibodies linked to a growth-inhibitory agent, and pharmaceutical compositions comprising antibodies or immunoconjugates of the invention. The invention also provides use of the antibodies, immunoconjugates and pharmaceutical compositions of the invention for the treatment of cancer or for diagnostic purposes.

### TECHNICAL FIELD

The present invention relates to antibodies which bind human GD2 (Disialoganglioside GD2), as well as to isolated nucleic acids and host cells comprising a sequence encoding said antibodies. The invention also relates to immunoconjugates comprising said antibodies linked to a growth-inhibitory agent, and to pharmaceutical compositions comprising the immunoconjugates of the invention. The invention also relates to the use of the antibodies, immunoconjugates and pharmaceutical compositions of the invention for the treatment of cancer and/or for diagnostic purposes.

### BACKGROUND

Antibody-drug conjugates (ADCs) are a class of therapeutics that combine the specificity of monoclonal antibodies (mAbs) with the potency of cytotoxic molecules. The use of ADC empowers the cancer killing activity of antibody by conjugated cytotoxic agents, while target-specific delivery reduces systemic toxicity caused by exposure to free toxic agents. Currently, a total of twelve ADCs have been approved by FDA for treating human cancers most of which were approved in last 2-3 years. The first two approved ADCs, ADCETRIS® (Brentuximab vedotin or SGN-35), an anti-CD30 antibody conjugated with cytotoxic agent MMAE, is designed to treat CD30-positive relapsing lymphoma and KADCYLA® (T-DM1), an anti-HER2 antibody conjugated with cytotoxic agent DM1, is designed to treat HER2-positive metastatic breast cancer. These initial approvals have been followed by at least eight more.

Linker technology profoundly impacts ADC potency, specificity, and safety. Enzyme-cleavable linkers utilize the differential activities of proteases inside and outside of the cells to achieve control of the drug release. A drug can be conjugated to antibody by a number of different linkers and can only be specifically cleaved by the action of lysosomal proteases present inside the cells, and at elevated levels in certain tumor types (Koblinsk et al, 2000). This will ensure the stability of linker in the blood stream to limit the damage to healthy tissue. However, the increased associated hydrophobicity of some enzyme-labile linkers can lead to aggregation of ADC, particularly with strongly hydrophobic drugs. Thus, there is a need for linkers which can provide serum stability, as well as increased solubility, allowing efficient conjugation and intracellular delivery of hydrophobic drugs.

The literature provides examples of anti-GD2 monoclonal antibodies in the treatment of neuroblastoma and other tumor indications. Today, three anti-GD2 naked antibody drugs have been approved for human use: naxitamab (Danyelza®) and dinutuximab (Unituxin®) in the United States, and dinutuximab beta (Qarziba®) in Europe. While dinutuximab and dinutuximab beta are mouse-human chimeric antibodies (ch14.18) produced in a mouse myeloma cell line designated SP2/0 and CHO (Chinese hamster ovarian) cells, respectively, naxitamab is a humanized antibody (hu3F8) produced by CHO cells.

Approximately 12% of all pediatric cancer patients succumb to neuroblastoma, the most common extracranial solid tumor of childhood. The majority of patients is diagnosed with high-risk neuroblastoma with a mortality of 50%. High-risk neuroblastoma therapy consists of multimodality treatment which are associated with severe short- and long-term toxicities. Recurrence rates are high with limited further treatment options. Intensification of conventional therapy has not improved outcome and is even associated with increased toxicity.

As noted above, the antibody dinutuximab, directed against ganglioside GD2, a carbohydrate-containing sphingolipid antigen uniformly expressed on neuroblastoma, cancer types of neuroectoderm origin, and neural tissue, was FDA-approved for neuroblastoma treatment. Application of this antibody, combined with cytokines and differentiation factors, has improved patient prognosis and demonstrated that neuroblastoma is susceptible to immunotherapy (Yu et al., 2010; New Engl. J. Med, Vol 363: pp 1324-34; and Suzuki and Cheung., 2015; Expert Opin Ther Targets Vol 19: p. 349-62). Dinutuximab improved event-free survival in comparison to the former standard chemotherapeutic treatments. Nevertheless, the positive clinical outcomes observed with the administration of anti-GD2 antibodies alone are associated with sometimes severe toxicities. While a host of toxicities have been documented (e.g., tachycardia, hypertension, hypotension, fever and urticaria) by far the most debilitating toxicity is neuropathic pain. Indeed despite co-administration of potent analgesics (including opioids), this neuropathic pain often limits the dose and, thereby, the efficacy of anti-GD2 antibodies. This neuropathic pain is likely mediated by the complement-dependent cytotoxicity (CDC) and antibody-dependent cell-mediated cytotoxicity (ADCC).

Moreover, progress has been made with the introduction of disialoganglioside (GD2) immunotherapy of patients suffering from high-risk neuroblastoma (from 12-month or above) or refractory/relapsed neuroblastoma (Ahmed et al., 2014, Nazha et al., 2020). However, neuropathic pain is a common and key dose-limiting adverse event hampering the full treatment potential for patients with the currently marketed GD2 antibodies (dinutuximab, naxitamab). It appears binding of anti-GD2 Ab to GD2 expressed on peripheral somatosensory and visceral nerves mediates intense, acute pain and/or allodynia in various body regions and is associated with peripheral nerve injury in patients (Yuki et al., 1997) and monkeys (EMA/263814/2017, 2017). Despite the relatively low expression of GD2 on peripheral nerves (Slart et al., 1997, Lammie et al., 1993, Yuki et al., 1997, Vriesendorp et al., 1997), it's likely the Fc-part effector functions of the Ab such as antibody-dependent cell-mediated cytotoxicity (ADCC) and complement-dependent cytotoxicity (CDC), that should also kill the tumor cells, triggers an immunological attack by humoral and cellular mediators to peripheral nerves that is manifested by neuropathy and acute neuropathic pain (Sorkin 2002, Dobrenkov and Cheung 2014; Mastrangelo et al., 2020, Nazha et al., 2020).

Another example of an ADC inducing peripheral neuropathy is monomethyl auristatin E (MMAE), a potent anti-cancer microtubule-targeting agent (MTA), used as a payload in three approved MMAE-containing ADCs (and, multiple ADCs currently in clinical development) to treat different types of cancers. MMAE-ADCs often induce peripheral neuropathy, a frequent adverse event leading to treatment dose reduction or discontinuation and subsequent clinical termination of many MMAE-ADCs. MMAE-ADC-induced peripheral neuropathy is attributed to non-specific uptake of the ADC in peripheral nerves and release of MMAE, disrupting microtubules (MTs) and causing neurodegeneration.

What is needed, therefore, is an anti-GD2 ADC which antibody, preferably reduced CDC and ADCC, conjugated to a non-neurotoxic cytotoxic payload (due in part to its chemical configuration and formulation) may more selectively deliver its cytotoxic payload to malignant cells without triggering the toxicities and, especially, the peripheral neuropathies which otherwise limit dosing and therapeutic efficacy.

### SUMMARY OF THE INVENTION

Humanized anti-GD2 antibody hu14.18 binds to the disialoganglioside GD2 that is found on the cell surface of tumors, including neuroblastoma, melanoma, sarcoma and others, and on normal tissues, primarily restricted to the central and peripheral nervous system in humans. Expression of disialoganglioside GD2 in normal tissues is limited essentially to the central nervous system, peripheral sensory nerve fibers, dermal melanocytes, lymphocytes, and mesenchymal stem cells. Treatment with anti-GD2 antibody can induce peripheral neuropathy and pain, which is thought to be related to antibody effector functions such as complement fixation and complement-dependent cytotoxicity (CDC) impacting peripheral nerves. Previously hu14.18 antibody was developed with the point mutation K322A (Kabat EU index numbering) in the immunoglobulin IgG1 constant domains, designated as “hu14.18-IgG1 (K322A)” (described in U.S. Pat. No. 8,835,606 B2, herein, incorporated by reference). This K322A point mutation in hu14.18 antibody was designed to prevent activation of the complement cascade and CDC effector functions, while still maintaining potential for antibody dependent cell-mediated cytotoxicity (ADCC) effector functions. In addition hu14.18-IgG1 (K322A) was produced with low fucosylation to enhance ADCC-activity (as described in U.S. Pat. No. 8,835,606 B2 all amino acid and nucleic acid sequences, herein, incorporated by reference). The ADCC-enhanced hu14.18-IgG1 (K322A) may induce less complement-fixation/CDC-mediated pain, while killing tumor cells via an ADCC mechanism of action. ADCC-enhanced hu14.18-IgG1 (K322A) is in clinical development (phase 2), but has not been approved. A chimeric anti-GD2 antibody with wild-type IgG1 isotype (dinutuximab) was approved for a defined set of pediatric neuroblastoma patients, and neuropathic pain associated with this treatment is known to occur and can require co-administration with pain relief agents.

In some embodiments, the present invention describes anti-GD2 hu14.18 antibody as an ADC with both ADCC and CDC antibody effector functions reduced or absent, thereby, providing the benefit of a larger therapeutic window to treat unmet medical needs. Designing hu14.18 antibodies without significant ADCC or CDC effector functions may reduce the risk of neuropathic pain induced by administration of anti-GD2 hu14.18 antibodies, while the conjugation of a drug to the antibody provides a different mechanism of action for such an anti-GD2-ADC to eliminate tumor cells. The combination of anti-GD2 hu14.18 lacking significant ADCC and CDC effector functions that delivers a potent conjugated drug as an ADC improves existing treatments and is likely to fill the (as of yet) unmet medical needs of patients. In selected embodiments, the selected cytotoxic drug should be free of neurotoxic properties.

Binding of an antibody to a specific antigen target is a property of the antigen-binding fragment (Fab) domain, including the specific light chain and heavy chain variable region complementarity-determining regions (CDR) amino acid sequences. Antibody effector functions and binding to Fc gamma receptors (FcγR) and neonatal Fc receptor (FcRn) are properties of the immunoglobulin (Ig) constant domains. There are different immunoglobulin constant domain genes, and these constant domain genes fused together with the Fab domain sequence determine the isotype of the antibody (IgM, IgG1, IgG2, etc.). In naturally occurring or recombinant antibodies the same antigen-binding domain amino acid sequences may be expressed fused to different constant domain isotypes, to produce antibodies with the same antigen target-binding specificity, but with different effector function properties. The abilities of an antibody to mediate activation of components of the immune system are termed the “effector functions” of the antibody, and important effector functions are mediated through binding to FcγR on immune cells and to complement proteins such as complement component 1q (C1q) in blood and extracellular fluids. Depending on the isotype an antibody links the specific antigen target on a cell to the immune system and can induce directed attack on the target cell by immune functions, such as Antibody-Dependent Cell-mediated Cytotoxicity (ADCC) and Complement-Dependent Cytotoxicity (CDC). Binding of the Ig constant domains to FcRn plays an important role in the in vivo half-life of an antibody, through increased recycling back to the circulation after an antibody has been internalized into cells. There have been a large number of investigations to test the role of specific amino acid residues in antibody constant domains for antibody effector functions and binding of antibodies to FcγR and complement proteins. Some changes in the amino acid sequence of human Ig constant domains may be used to produce recombinant antibodies with altered properties for antibody effector functions and binding of antibodies to FcγR and complement proteins. In selected embodiments, the present invention is to provides ADC proteins that bind the target GD2 to deliver ADC molecules to cells and without significant antibody effector functions.

In some embodiments of the present invention, the production and use of an anti-GD2 hu14.18-IgG1.4 (K322A)-delK antibody conjugated to a small molecule toxic payload from the class of Exatecan topoisomerase-I inhibitors for the treatment of different tumor indications, e.g. sarcomas, neuroblastoma and SCLC is described. This novel ADC offers a preferred therapeutic window as compared to other GD2 antibody therapies already in clinical use.

In preferred embodiments of the present invention, the Fc-part effector functionality was engineered out of the ADC and, instead, the non-neurotoxic payload Exatecan was used to kill tumor cells with a resulting absence of PNS injury and obvious clinical pain signals in rats and monkeys after repeated infusions of Molecule 1.

### DETAILED DESCRIPTION OF THE INVENTION

### Definitions

“GD2” is a disialoganglioside expressed on tumors of neuroectodermal origin, including human neuroblastoma and melanoma, with highly restricted expression on normal tissues, principally to the central and peripheral nervous system in humans. GD2 is defined by the following chemical structure:

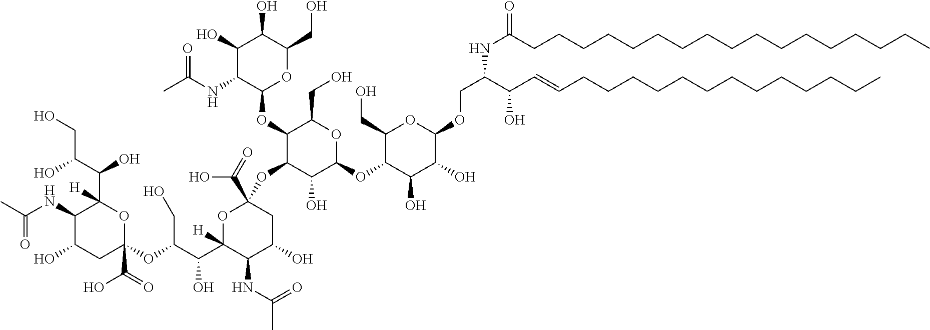 [SMILES: CCCCCCCCCCCCC/C=C/[C@H](O)[C@H](CO[C@@H]1O[C@H](CO)[C@@H](O[C@@H]2O[C@H](CO)[C@H](O[C@@H]3O[C@H](CO)[C@H](O)[C@H](O)[C@H]3NC(C)=O)[C@H](O[C@]3(C(=O)O)C[C@H](O)[C@@H](NC(C)=O)[C@H]([C@H](O)[C@@H](CO)O[C@]4(C(=O)O)C[C@H](O)[C@@H](NC(C)=O)[C@H]([C@H](O)[C@H](O)CO)O4)O3)[C@H]2O)[C@H](O)[C@H]1O)NC(=O)CCCCCCCCCCCCCCCCC]

Which corresponds to the following IUPAC name:
(2R,4R,5S,6S)-2-[3-[(2S,3S,4R,6S)-6-[(2S,3R,4R,5S,6R)-5-[(2S,3R,4R,5R,6R)-3- acetamido-4,5-dihydroxy-6-(hydroxymethyl) oxan-2-yl]oxy-2-[(2R,3S,4R,5R,6R)-4,5-dihydroxy-2-(hydroxymethyl)-6-[(E)-3-hydroxy-2-(octadecanoylamino) octadec-4-enoxy]oxan-3-yl]oxy-3-hydroxy-6-(hydroxymethyl) oxan-4-yl]oxy-3-amino-6-carboxy-4-hydroxyoxan-2-yl]-2,3-dihydroxypropoxy]-5-amino-4-hydroxy-6-(1,2,3-trihydroxypropyl) oxane-2-carboxylic acid.

As used, herein, the designation “M4344” refers to an adenosine triphosphate (ATP)-competitive inhibitor having the following chemical structure:

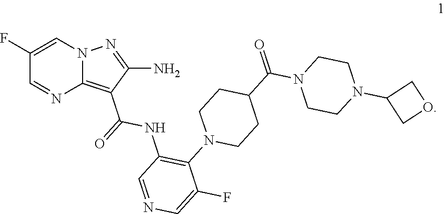 [SMILES: Nc1nn2cc(F)cnc2c1C(=O)Nc1cncc(F)c1N1CCC(C(=O)N2CCN(C3COC3)CC2)CC1]

A “domain” or “region” may be any region of a protein, generally defined on the basis of sequence homologies and often related to a specific structural or functional entity. GD2 family members are known to be composed of Ig-like domains. The term domain is used in this document to designate either individual Ig-like domains, such as “N-domain” or for groups of consecutive domains, such as “A2-B2 domain”.

A “coding sequence” or a sequence “encoding” an expression product, such as a polypeptide, protein, or enzyme, is a nucleotide sequence that, when expressed, results in the production of that polypeptide, protein, or enzyme, i.e., the nucleotide sequence encodes an amino acid sequence for that polypeptide, protein or enzyme. A coding sequence for a protein may include a start codon (usually ATG) and a stop codon.

As used herein, references to specific proteins (e.g. antibodies) can include a polypeptide having a native amino acid sequence, as well as variants and modified forms regardless of their origin or mode of preparation. A protein which has a native amino acid sequence is a protein having the same amino acid sequence as obtained from nature. Such native sequence proteins can be isolated from nature or can be prepared using standard recombinant and/or synthetic methods. Native sequence proteins specifically encompass naturally occurring truncated or soluble forms, naturally occurring variant forms (e.g. alternatively spliced forms), naturally occurring allelic variants and forms including post-translational modifications. Native sequence proteins include proteins carrying post-translational modifications such as glycosylation, or phosphorylation, or other modifications of some amino acid residues.

The term “gene” means a DNA sequence that codes for, or corresponds to, a particular sequence of amino acids which comprises all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription. In particular, the term gene may be intended for the genomic sequence encoding a protein, i.e. a sequence comprising regulator, promoter, intron and exon sequences.

Herein, a sequence “at least 85% identical” to a reference sequence is a sequence having, over its entire length, 85% or more, for instance 90%, 91%, 92%, 93%, 94%, 95%, 96%, 97%, 98% or 99% sequence identity with the entire length of the reference sequence. The percentage of “sequence identity” may thus be determined by comparing two such sequences over their entire length by global pairwise alignment using the algorithm of Needleman and Wunsch (J. Mol. Biol. 48:443 (1970)), e.g. using the program Needle (EMBOSS) with the BLOSUM62 matrix and the following parameters: gap open=10, gap extend=0.5, end gap penalty=false, end gap open=10, end gap extend=0.5 (which are standard settings).

A “conservative amino acid substitution” is one in which an amino acid residue is substituted by another amino acid residue having a side chain with similar chemical properties (e.g., charge, size or hydrophobicity). In general, a conservative amino acid substitution will not substantially change the functional properties of a protein. Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Conservative amino acid substitution groups can also be defined on the basis of amino acid size.

An “antibody” (also referred to as an “immunoglobulin”) may e.g. be a natural or conventional type of antibody in which two heavy chains are linked to each other by disulfide bonds and each heavy chain is linked to a light chain by a disulfide bond. There are two types of light chain, lambda (I) and kappa (κ). There are five main heavy chain classes (or isotypes) which determine aspects of the functional activity of an antibody molecule: IgM, IgD, IgG, IgA and IgE. Each antibody chain contains distinct sequence domains (or regions). The light chain of a typical IgG antibody includes two regions, a variable region (VL) and a constant region (CL). The heavy chain of a typical IgG antibody includes four regions, namely a variable region (VH) and a constant region (CH), the latter being made up of three constant domains (CH1, CH2 and CH3). The variable regions of both light and heavy chains determine binding and specificity to the antigen. The constant regions of the light and heavy chains can confer important biological properties, such as antibody chain association, secretion, trans-placental mobility, complement binding, and binding to Fc receptors (FcR). The Fv fragment is the N-terminal part of the Fab fragment of an antibody and consists of the variable portions of one light chain and one heavy chain.

The specificity of the antibody resides in the structural complementarity between the antibody combining site and the antigenic determinant. Antibody combining sites are made up of residues that are primarily from the so-called hypervariable or complementarity determining regions (CDRs). Complementarity determining regions (CDRs) therefore refer to amino acid sequences which together define the binding affinity and specificity of the Fv region of an antibody. The light (L) and heavy (H) chains of an antibody each have three CDRs, designated CDR1-L, CDR2-L, CDR3-L and CDR1-H, CDR2-H, CDR3-H, respectively. A conventional antibody's antigen-binding site, therefore, includes six CDRs, comprising the CDR set from each of a heavy and a light chain variable region.

“Framework regions” (FRs) refer to amino acid sequences interposed between CDRs, i.e. to those portions of immunoglobulin light and heavy chain variable regions that are relatively conserved among different immunoglobulins in a single species. The light and heavy chains of an immunoglobulin each have four FRs, designated FR1-L, FR2-L, FR3-L, FR4-L, and FR1-H, FR2-H, FR3-H, FR4-H, respectively. As used herein, a “human framework region” is a framework region that is substantially identical (about 85%, or more, for instance 90%, 91%, 92%, 93%, 94%, 95%, 96%, 97%, 98% or 99%) to the framework region of a naturally occurring human antibody.

The term “monoclonal antibody” or “mAb” as used herein refers to an antibody molecule of a single amino acid sequence, which is directed against a specific antigen, and is not to be construed as requiring production of the antibody by any particular method. A monoclonal antibody may be produced e.g. by a single clone of B cells or hybridoma, but may also be recombinant, e.g. produced by methods involving genetic or protein engineering.

The term “chimeric antibody” refers to an engineered antibody which, in its broadest sense, contains one or more regions from one antibody and one or more regions from one or more other antibodies. In an embodiment, a chimeric antibody comprises a VH and a VL of an antibody derived from a non-human animal, in association with a CH and a CL of another antibody which is, in some embodiments, a human antibody. As the non-human animal, any animal such as mouse, rat, hamster, rabbit or the like can be used. A chimeric antibody may also denote a multispecific antibody having specificity for at least two different antigens.

The term “humanized antibody” refers to an antibody which is wholly or partially of non-human origin and which has been modified to replace certain amino acids, for instance in the framework regions of the VH and VL, in order to avoid or minimize an immune response in humans. The constant regions of a humanized antibody are typically human CH and CL regions.

“Fragments” of antibodies (e.g. of conventional antibodies) comprise a portion of an intact antibody such as an IgG, in particular an antigen binding region or variable region of the intact antibody. Examples of antibody fragments include Fv, Fab, F(ab′) 2, Fab′, dsFv, (dsFv) 2, scFv, sc (Fv) 2, diabodies, as well as bispecific and multispecific antibodies formed from antibody fragments. A fragment of a conventional antibody may also be a single domain antibody, such as a heavy chain antibody or VHH.

The term “Fab” denotes an antibody fragment having a molecular weight of about 50,000 Da and antigen binding activity, in which about a half of the N-terminal side of the heavy chain and the entire light chain are bound together through a disulfide bond. It is usually obtained among fragments by treating IgG with a protease, papain.

The term “F(ab′) 2” refers to an antibody fragment having a molecular weight of about 100,000 Da and antigen binding activity, which is slightly larger than 2 identical Fab fragments bound via a disulfide bond of the hinge region. It is usually obtained among fragments by treating IgG with a protease, pepsin.

The term “Fab′” refers to an antibody fragment having a molecular weight of about 50,000 Da and antigen binding activity, which is obtained by cutting a disulfide bond of the hinge region of the F(ab′) 2.

A single chain Fv (“scFv”) is a covalently linked VH::VL heterodimer which is usually expressed from a gene fusion including VH and VL encoding genes linked by a peptide-encoding linker. The human scFv fragments of the invention include CDRs that are held in appropriate conformation, for instance by using gene recombination techniques. Divalent and multivalent antibody fragments can form either spontaneously by association of monovalent scFvs, or can be generated by coupling monovalent scFvs by a peptide linker, such as divalent sc (Fv) 2. “dsFv” is a VH::VL heterodimer stabilized by a disulphide bond. “(dsFv)2” denotes two dsFv coupled by a peptide linker.

An “antibody-drug conjugate”, or “ADC” is an antibody that is conjugated to one or more cytotoxins, each through a linker. The antibody is typically a monoclonal antibody specific to an antigen. In certain preferred embodiments of the present invention, ADCs are designed as a targeted therapy for treating cancer.

Unlike chemotherapy alone, these preferred embodiments combine the targeting capabilities of monoclonal antibodies with the cancer-killing ability of cytotoxic drugs and may discriminate between healthy and malignant tissue.

As used, herein, “Molecule 1” (also referred to, herein, as “M3554”) refers to the following ADC:

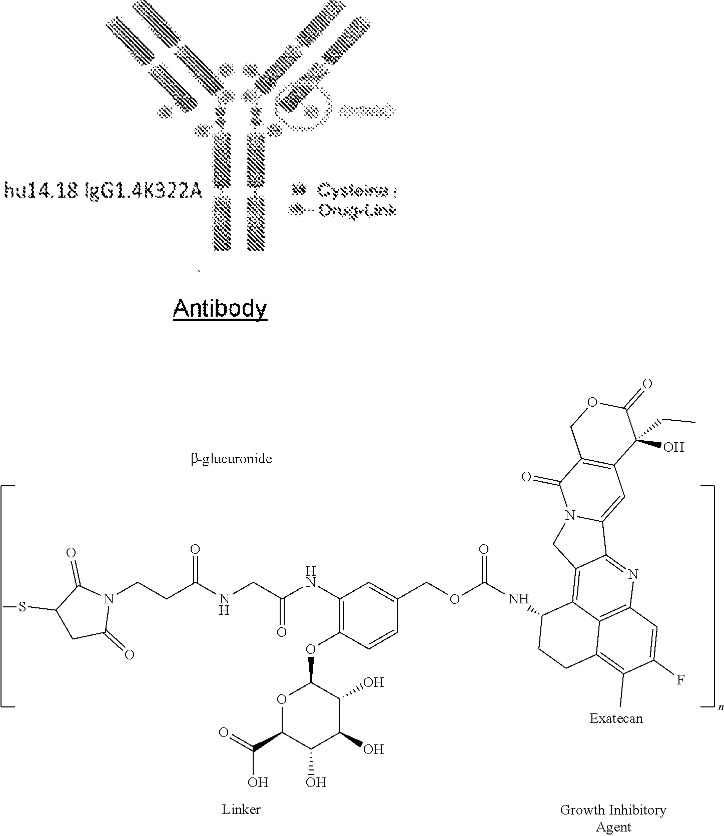

wherein: the antibody binds to GD2, said antibody comprising the amino acid sequence of SEQ ID NO:4 and the amino acid sequence of SEQ ID NO: 1, the linker is β-glucuronide, and the growth inhibitory agent is Exatecan.

The term “hybridoma” denotes a cell, which is obtained by subjecting a B cell prepared by immunizing a non-human mammal with an antigen to cell fusion with a myeloma cell derived from a mouse or the like which produces a desired monoclonal antibody having an antigen specificity. In some examples, the designation “M3554” is synonymous with Molecule 1.

By “purified” or “isolated” it is meant, when referring to a polypeptide (e.g. an antibody) or a nucleotide sequence, that the indicated molecule is present in the substantial absence of other biological macromolecules of the same type. The term “purified” as used herein means at least 75%, 85%, 95%, 96%, 97%, or 98% by weight, of biological macromolecules of the same type are present. An “isolated” nucleic acid molecule which encodes a particular polypeptide refers to a nucleic acid molecule which is substantially free of other nucleic acid molecules that do not encode the subject polypeptide; however, the molecule may include some additional bases or moieties which do not deleteriously affect the basic characteristics of the composition.

As used herein, the term “subject” denotes a mammal, such as a rodent, a feline, a canine, a primate or a human. In embodiments of the invention, the subject (or patient) is a human.

As used herein, the term “peripheral neuropathy” denotes the damage to the nerves located outside of the brain and spinal cord (peripheral nerves) clinically manifesting in weakness, numbness and/or pain.

As used herein, “neuropathic pain” refers to pain caused by damage or injury to the nerves that transfer information between the brain and spinal cord from the skin, muscles and other parts of the body clinically manifesting as a burning sensation with affected areas often sensitive to the touch.

### Methods of Producing Antibodies of the Invention

Antibodies of the invention may be produced by any technique known in the art, such as, without limitation, any chemical, biological, genetic or enzymatic technique, either alone or in combination.

Knowing the amino acid sequence of a desired antibody, one skilled in the art can readily produce said antibodies or immunoglobulin chains using standard techniques for production of polypeptides. For instance, they can be synthesized using well-known solid phase methods using a commercially available peptide synthesis apparatus (such as that made by Applied Biosystems, Foster City, California) and following the manufacturer's instructions. Alternatively, antibodies and immunoglobulin chains of the invention can be produced by recombinant DNA techniques, as is well-known in the art. For example, these polypeptides (e.g. antibodies) can be obtained as DNA expression products after incorporation of DNA sequences encoding the desired polypeptide into expression vectors and introduction of such vectors into suitable eukaryotic or prokaryotic hosts that will express the desired polypeptide, from which they can be later isolated using well-known techniques.

Amino Acid sequences of antibody chains used to produce the protein precursors of anti-GD2-ADC molecules. Mature anti-GD2 hu14.18 light chain and heavy chain amino acid sequences are shown. The C-terminus of the hu14.18 heavy chains may end either as shown with G446 (Kabat EU index numbering) or with K447 (Kabat EU index numbering). Changes in constant domains from wild-type human IgG1 sequences are underlined.

```
hu14.18 mature light chain amino acid sequence:
  (SEQ ID NO: 1)
DWVMTQTPLSLPVTPGEPASISCRSSQSLVHRNGNTYLHWYLQKPGQSPKLLIHKVSNRFS
GVPDRFSGSGSGTDFTLKISRVEAEDLGVYFCSQSTHVPPLTFGAGTKLELKRTVAAPSVFI
FPPSDEQLKSGTASWVCLLNNFYPREAKVQWKVDNALQSGNSQESVTEQDSKDSTYSLSS
TLTLSKADYEKHKVYACEVTHQGLSSPVTKSFNRGEC
hu14.18-IgG1-delK mature heavy chain amino acid sequence:
  (SEQ ID NO: 2)
EVQLVQSGAEVEKPGASVKISCKASGSSFTGYNMNWVRQNIGKSLEWIGAIDPYYGGTSYN
QKFKGRATLTVDKSTSTAYMHLKSLRSEDTAVYYCVSGMEYWGQGTSVTVSSASTKGPSV
FPLAPSSKSTSGGTAALGCLVKDYFPEPVTVSWNSGALTSGVHTFPAVLQSSGLYSLSSVV
TVPSSSLGTQTYICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHE
ALHNHYTQKSLSLSPG
hu14.18-IgG1.4-delK mature heavy chain amino acid sequence:
  (SEQ ID NO: 3)
EVQLVQSGAEVEKPGASVKISCKASGSSFTGYNMNWVRQNIGKSLEWIGAIDP
YYGGTSYNQKFKGRATLTVDKSTSTAYMHLKSLRSEDTAVYYCVSGMEYWGQGTSVTVSS
ASTKGPSVFPLAPSSKSTSGGTAALGCLVKDYFPEPVTVSWNSGALTSGVHTFPAVLQSSG
LYSLSSWVTVPSSSLGTQTYICNVNHKPSNTKVDKRVEPKSCDKTHTCPPCPAPPVAGPSVF
LFPPKPKDTLMISRTPEVTCWVDVSHEDPEVKFNWYVDGVEVHNAKTKPREEQYNSTYRV
VSVLTVLHQDWLNGKEYKCKVSNKALPSSIEKTISKAKGQPREPQVYTLPPSREEMTKNQV
SLTCLVKGFYPSDIAVEWESNGQPENNYKTTPPVLDSDGSFFLYSKLTVDKSRWQQGNVFS
CSVMHEALHNHYTQKSLSLSPG
hu14.18-IgG1.4(K322A)-delK heavy chain amino acid sequence
  (SEQ ID NO: 4)
EVQLVQSGAEVEKPGASVKISCKASGSSFTGYNMNWVRQNIGKSLEWIGAIDPYYGGTSYN
QKFKGRATLTVDKSTSTAYMHLKSLRSEDTAVYYCVSGMEYWGQGTSVTVSSASTKGPSV
FPLAPSSKSTSGGTAALGCLVKDYFPEPVTVSWNSGALTSGVHTFPAVLQSSGLYSLSSVV
TVPSSSLGTQTYICNVNHKPSNTKVDKRVEPKSCDKTHTCPPCPAPPVAGPSVFLFPPKPK
DTLMISRTPEVTCVVVDVSHEDPEVKFNWYVDGVEVHNAKTKPREEQYNSTYRVVSVLTVL
HQDWLNGKEYKCAVSNKALPSSIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHE
ALHNHYTQKSLSLSPG
```

Flow chart providing the candidate characterization cascade used to test the anti-GD2 antibody protein precursor proteins:

The invention further relates to a method of producing an antibody of the invention, which method comprises the steps consisting of: (i) culturing a transformed host cell according to the invention; (ii) expressing the antibody; and (iii) recovering the expressed antibody.

Antibodies of the invention can be suitably separated from the culture medium by conventional immunoglobulin purification procedures such as, for example, protein A-Sepharose, hydroxyapatite chromatography, gel electrophoresis, dialysis, or affinity chromatography.

In some embodiments, a humanized chimeric antibody of the present invention can be produced by obtaining nucleic acid sequences encoding humanized VL and VH regions as previously described, constructing a human chimeric antibody expression vector by inserting them into an expression vector for animal cell having genes encoding human antibody CH and human antibody CL, and expressing the coding sequence by introducing the expression vector into an animal cell.

As the CH domain of a human chimeric antibody, any region which belongs to human immunoglobulin heavy chains may be used, for instance those of IgG class are suitable and any one of subclasses belonging to IgG class, such as IgG1, IgG2, IgG3 and IgG4, can be used. Also, as the CL of a human chimeric antibody, any region which belongs to human immunoglobulin light chains may be used, and those of kappa class or lambda class can be used.

Methods for producing humanized or chimeric antibodies may involve conventional recombinant DNA and gene transfection techniques are well known in the art (see e.g. Morrison S L. et al. (1984) and patent documents U.S. Pat. Nos. 5,202,238; and 5,204,244).

Methods for producing humanized antibodies based on conventional recombinant DNA and gene transfection techniques are well known in the art (see, e. g., Riechmann L. et al. 1988; Neuberger M S. et al. 1985). Antibodies can be humanized using a variety of techniques known in the art including, for example, the technique disclosed in the application WO2009/032661, CDR-grafting (EP 239,400; PCT publication WO91/09967; U.S. Pat. Nos. 5,225,539; 5,530,101; and 5,585,089), veneering or resurfacing (EP 592, 106; EP 519,596; Padlan E A (1991); Studnicka G M et al. (1994); Roguska M A. et al. (1994)), and chain shuffling (U.S. Pat. No. 5,565,332). The general recombinant DNA technology for preparation of such antibodies is also known (see European Patent Application EP 125023 and International Patent Application WO 96/02576).

A Fab of the present invention can be obtained by treating an antibody of the invention (e.g. an IgG) with a protease, such as papain. Also, the Fab can be produced by inserting DNA sequences encoding both chains of the Fab of the antibody into a vector for prokaryotic expression, or for eukaryotic expression, and introducing the vector into prokaryotic or eukaryotic cells (as appropriate) to express the Fab.

A F(ab′) 2 of the present invention can be obtained treating an antibody of the invention (e.g. an IgG) with a protease, pepsin. Also, the F(ab′) 2 can be produced by binding a Fab′ described below via a thioether bond or a disulfide bond.

A Fab′ of the present invention can be obtained by treating F(ab′) 2 of the invention with a reducing agent, such as dithiothreitol. Also, the Fab′ can be produced by inserting DNA sequences encoding Fab′ chains of the antibody into a vector for prokaryotic expression, or a vector for eukaryotic expression, and introducing the vector into prokaryotic or eukaryotic cells (as appropriate) to perform its expression.

A scFv of the present invention can be produced by taking sequences of the CDRs or VH and VL domains as previously described for the antibody of the invention, then constructing a DNA encoding a scFv fragment, inserting the DNA into a prokaryotic or eukaryotic expression vector, and then introducing the expression vector into prokaryotic or eukaryotic cells (as appropriate) to express the scFv. To generate a humanized scFv fragment, a well-known technology called CDR grafting may be used, which involves selecting the complementary determining regions (CDRs) according to the invention, and grafting them onto a human scFv fragment framework of known three dimensional structure (see, e. g., WO98/45322; WO 87/02671; U.S. Pat. Nos. 5,859,205; 5,585,089; 4,816,567; EP0173494).

### Modified Anti-GD2 Antibodies

Amino acid sequence modification(s) of the antibodies described herein are contemplated. For example, it may be desirable to improve the binding affinity and/or other biological properties of the antibody.

Modifications and changes may be made in the structure of the antibodies of the present invention, and in the DNA sequences encoding them, and still result in a functional antibody or polypeptide with desirable characteristics.

In making the changes in the amino sequences of polypeptide, the hydropathic index of amino acids may be considered. The importance of the hydropathic amino acid index for the interactive biologic function of a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

A further aspect of the present invention also encompasses function-conservative variants of the polypeptides of the present invention.

For example, certain amino acids may be substituted by other amino acids in a protein structure without appreciable loss of activity. Since the interactive capacity and nature of a protein define its biological functional activity, certain amino acid substitutions can be made in a protein sequence, and of course in its encoding DNA sequence, while nevertheless obtaining a protein with like properties. It is thus contemplated that various changes may be made in the antibody sequences of the invention, or corresponding DNA sequences which encode said polypeptides, without appreciable loss of their biological activity.

It is known in the art that certain amino acids may be substituted by other amino acids having a similar hydropathic index or score and still result in a protein with similar biological activity, i.e. still obtain a biological functionally equivalent protein. It is also possible to use well-established technologies, such as alanine-scanning approaches, to identify, in an antibody or polypeptide of the invention, all the amino acids that can be substituted without significant loss of binding to the antigen. Such residues can be qualified as neutral, since they are not involved in antigen binding or in maintaining the structure of the antibody. One or more of these neutral positions can be substituted by alanine or by another amino acid can without changing the main characteristics of the antibody or polypeptide of the invention.

Neutral positions can be seen as positions where any amino acid substitution could be incorporated. Indeed, in the principle of alanine-scanning, alanine is chosen since it this residue does not carry specific structural or chemical features. It is generally admitted that if an alanine can be substituted for a specific amino acid without changing the properties of a protein, many other, if not all amino acid substitutions are likely to be also neutral. In the opposite case where alanine is the wild-type amino acid, if a specific substitution can be shown as neutral, it is likely that other substitutions would also be neutral.

As outlined above, amino acid substitutions are generally based on the relative similarity of the amino acid side-chain substituents, for example, their hydrophobicity, hydrophilicity, charge, size, and the like. Exemplary substitutions which take any of the foregoing characteristics into consideration are well known to those of skill in the art and include: arginine and lysine; glutamate and aspartate; serine and threonine; glutamine and asparagine; and valine, leucine and isoleucine.

It may be also desirable to modify the antibody of the invention with respect to effector function, e.g. so as to enhance antigen-dependent cell-mediated cytotoxicity (ADCC) and/or complement dependent cytotoxicity (CDC) of the antibody, or e.g. to alter the binding to Fc receptors. This may be achieved by introducing one or more amino acid substitutions in an Fc region of the antibody. Alternatively or additionally, cysteine residue(s) may be introduced in the Fc region, thereby allowing inter-chain disulfide bond formation in this region. The homodimeric antibody thus generated may have improved internalization capability and/or increased complement-mediated cell killing and/or antibody-dependent cellular cytotoxicity (ADCC) (Caron P C. et al. 1992; and Shopes B. 1992). In some embodiments, an antibody of the invention may be an antibody with a modified amino acid sequence that results in reduced or eliminated binding to most Fcγ receptors, which can reduce uptake and toxicity in normal cells and tissues expressing such receptors, e.g. macrophages, liver sinusoidal cells etc.

Another type of amino acid modification of the antibody of the invention may be useful for altering the original glycosylation pattern of the antibody, i.e. by deleting one or more carbohydrate moieties found in the antibody, and/or adding one or more glycosylation sites that are not present in the antibody. The presence of either of the tripeptide sequences asparagine-X-serine, and asparagine-X-threonine, where X is any amino acid except proline, creates a potential glycosylation site. Addition or deletion of glycosylation sites to the antibody can conveniently be accomplished by altering the amino acid sequence such that it contains one or more of the above-described tripeptide sequences (for N-linked glycosylation sites).

Another type of modification involves the removal of sequences identified, either in silico or experimentally, as potentially resulting in degradation products or heterogeneity of antibody preparations. As examples, deamidation of asparagine and glutamine residues can occur depending on factors such as pH and surface exposure. Asparagine residues are particularly susceptible to deamidation, primarily when present in the sequence Asn-Gly, and to a lesser extent in other dipeptide sequences such as Asn-Ala. When such a deamidation site, in particular Asn-Gly, is present in an antibody or polypeptide, it may therefore be considered to remove the site, typically by conservative substitution to remove one of the implicated residues. Such substitutions in a sequence to remove one or more of the implicated residues are also intended to be encompassed by the present invention.

Another type of covalent modification involves chemically or enzymatically coupling glycosides to the antibody. These procedures are advantageous in that they do not require production of the antibody in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, orhydroxyproline, (e) aromatic residues such as those of phenylalanine, tyrosine, or tryptophan, or (f) the amide group of glutamine. For example, such methods are described in WO87/05330.

Removal of carbohydrate moieties present on the antibody may be accomplished chemically or enzymatically. Chemical deglycosylation requires exposure of the antibody to the compound trifluoromethanesulfonic acid, or an equivalent compound. This treatment results in the cleavage of most or all sugars except the linking sugar (N-acetylglucosamine or N-acetylgalactosamine), while leaving the antibody intact. Chemical deglycosylation is described by Sojahr H. et al. (1987) and by Edge, A S. et al. (1981). Enzymatic cleavage of carbohydrate moieties on antibodies can be achieved by the use of a variety of endo- and exo-glycosidases as described by Thotakura, N R. et al. (1987).

Another type of covalent modification of the antibody comprises linking the antibody to one of a variety of non-proteinaceous polymers, e.g. polyethylene glycol, polypropylene glycol, or polyoxyalkylenes, e.g. in the manner set forth in U.S. Pat. Nos. 4,640,835; 4,496,689; 4,301,144; 4,670,417; 4,791,192 and 4,179,337.

Other amino acid sequence modifications known in the art may also be applied to an antibody of the invention.

### Anti-GD2 ADCs

The present invention provides immunoconjugates, also referred to herein as ADCs or, more briefly, conjugates. As used herein, all these terms have the same meaning and are interchangeable. The immunoconjugates of the present invention may be prepared according to in vitro methods as described herein.

The present invention provides an ADC comprising an antibody of the invention (such as e.g. mAb1, or an antibody with similar CDRs as mAb1) covalently linked via a linker to at least one growth inhibitory agent.

The term “growth inhibitory agent” (also referred to as an “anti-proliferative agent”) refers to a molecule or compound or composition which inhibits growth of a cell, such as a tumor cell, in vitro and/or in vivo.

In some embodiments, the growth inhibitory agent is a cytotoxic drug (also referred to as a cytotoxic agent). The present invention also contemplates radioactive moieties for use as cytotoxic drugs.

The term “cytotoxic drug” as used herein refers to a substance that directly or indirectly inhibits or prevents the function of cells and/or causes destruction of the cells. The term “cytotoxic drug” includes e.g. chemotherapeutic agents, enzymes, antibiotics, toxins such as small molecule toxins or enzymatically active toxins, toxoids, vincas, taxanes, maytansinoids or maytansinoid analogs, tomaymycin or pyrrolobenzodiazepine derivatives, cryptophycin derivatives, leptomycin derivatives, auristatin or dolastatin analogs, prodrugs, topoisomerase I inhibitors, topoisomerase II inhibitors, DNA alkylating agents, anti-tubulin agents, CC-1065 and CC-1065 analogs.

Topoisomerase I inhibitors are molecules or compounds that inhibit the human enzyme topoisomerase I which is involved in altering the topology of DNA by catalyzing the transient breaking and rejoining of a single strand of DNA. Topoisomerase I inhibitors are highly toxic to dividing cells e.g. of a mammal. Examples of suitable topoisomerase I inhibitors include camptothecin (CPT) and analogs thereof such as topotecan, irinotecan, silatecan, cositecan, Exatecan, lurtotecan, gimatecan, belotecan and rubitecan.

In some embodiments, the immunoconjugates of the invention comprise the cytotoxic drug Exatecan as the growth inhibitory agent. Exatecan has the IUPAC chemical name:
(1S,9S)-1-Amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione.

### Exatecan is Represented by the Following Structural Formula (I):

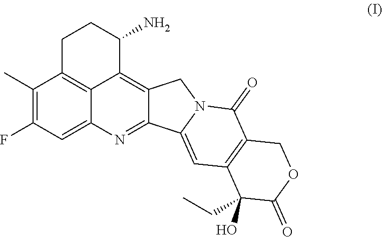 [SMILES: CC[C@@]1(O)C(=O)OCc2c1cc1n(c2=O)Cc2c-1nc1cc(F)c(C)c3c1c2[C@@H](N)CC3]

Exatecan is a modified derivative of camptothecin, with an additional alicyclic ring fused to rings A and B that bears a solubilizing primary amine (equivalent to a 7-CH2NH2 substituent on camptothecin). There are also lipophilic substituents at positions 10 and 11 on ring A that enhance membrane permeability.

In further embodiments of the invention, other CPT analogs and other cytotoxic drugs may be used, e.g. as listed above. Examples of some cytotoxic drugs and of methods of conjugation are further given in the application WO2008/010101 which is incorporated by reference.

The term “radioactive moiety” refers to a chemical entity (such as a molecule, compound or composition) that comprises or consists of a radioactive isotope suitable for treating cancer, such as At²¹¹, Bi²¹², Er¹⁶⁹, I¹³¹, I¹²⁵, Y⁹⁰, In¹¹¹, P³², Re¹⁸⁶, Re¹⁸, Sm¹⁵³, Sr⁸⁹, or radioactive isotopes of Lu. Such radioisotopes generally emit mainly beta-radiation. In some embodiments, the radioactive isotope is an alpha-emitter isotope, for example Thorium 227 which emits alpha-radiation. Immunoconjugates can be prepared e.g. as described in the application WO2004/091668.

In an immunoconjugate of the present invention, an antibody of the present invention is covalently linked via a linker to the at least one growth inhibitory agent. “Linker”, as used herein, means a chemical moiety comprising a covalent bond and/or any chain of atoms that covalently attaches the growth inhibitory agent to the antibody. Linkers are well known in the art and include e.g. disulfide groups, thioether groups, acid labile groups, photolabile groups, peptidase labile groups and esterase labile groups. Conjugation of an antibody of the invention with cytotoxic drugs or other growth inhibitory agents may be performed e.g. using a variety of bifunctional protein coupling agents including but not limited to N-succinimidyl pyridyldithiobutyrate (SPDB), butanoic acid 4-[(5-nitro-2-pyridinyl)dithio]-2,5-dioxo-1-pyrrolidinyl ester (nitro-SPDB), 4-(Pyridin-2-yldisulfanyl)-2-sulfo-butyric acid (sulfo-SPDB), N-succinimidyl (2-pyridyldithio) propionate (SPDP), succinimidyl (N-maleimidomethyl) cyclohexane-1-carboxylate (SMCC), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl)-hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al (1987). Carbon labeled 1-isothiocyanatobenzyl methyldiethylene triaminepentaacetic acid (MX-DTPA) is an exemplary chelating agent for conjugation of radionucleotide to an antibody (WO 94/11026).

In embodiments of the present invention, the linker may be a “cleavable linker”, which may facilitate release of the cytotoxic drug or other growth inhibitory agent inside of or in the vicinity of a cell, e.g. a tumor cell. In some embodiments, the linker is a linker cleavable in an endosome of a mammalian cell. For example, an acid-labile linker, a peptidase-sensitive linker, an esterase labile linker, a photolabile linker or a disulfide-containing linker (see e.g. U.S. Pat. No. 5,208,020) may be used.

When referring to a structural formula representing an immunoconjugate, the following nomenclature is also used herein: a growth inhibitory agent and a linker, taken together, are also referred to as a [(linker)-(growth inhibitory agent)] moiety; for instance, an Exatecan molecule and a linker, taken together, are also referred to as a [(linker)-(Exatecan)] moiety.

In some specific embodiments of the present invention, the linker is a linker cleavable by the human enzyme glucuronidase. For example, an immunoconjugate of the present invention may thus have the following formula (II) which includes a linker cleavable by glucuronidase:

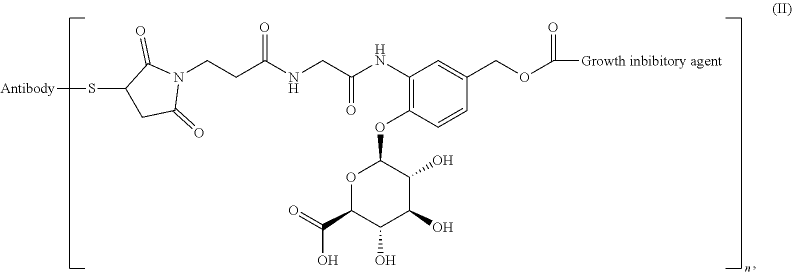

wherein the antibody is the antibody of the invention, wherein S is a sulfur atom of the antibody, and wherein n is a number of [(linker)-(growth inhibitory agent)] moieties covalently linked to the antibody. The number n may be e.g. between 1 and 10; in more specific embodiments, n is between 7 and 8; in even more specific embodiments, n is between 7.5 and 8.0 (i.e. about 8). In some embodiments, S is a sulfur atom of a cysteine of the antibody. In some embodiments, the antibody is mAb1.

The number n is also referred to as “drug-to-antibody ratio” (or “DAR”); this number n is always to be understood as an average number for any given (preparation of an) immunoconjugate.

In other specific embodiments of the present invention, the linker is a linker cleavable by the human enzyme legumain. For example, an immunoconjugate of the present invention may thus have the following formula (III) which includes a linker cleavable by legumain:

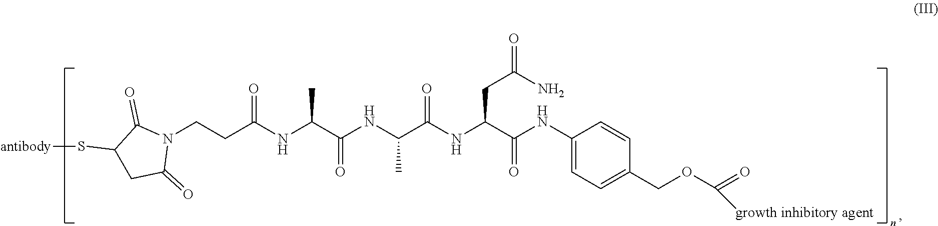

wherein the antibody is the antibody of the invention, wherein S is a sulfur atom of the antibody, and wherein n is a number of [(linker)-(growth inhibitory agent)] moieties covalently linked to the antibody. The number n (also referred to as the DAR) may be e.g. between 1 and 10; in more specific embodiments, n is between 7 and 8; in even more specific embodiments, n is between 7.5 and 8.0 (i.e. about 8). In some embodiments, S is a sulfur atom of a cysteine of the antibody. In some embodiments, the antibody is mAb1.

In each of the above formulae (II) and (III), the chemical structure between the sulfur atom of the antibody and the growth inhibitory agent is a linker. One of these linkers is also contained in each of the formulae (IV) to (IX) depicted further below.

In any one of the embodiments with linkers cleavable by glucuronidase or legumain, as described above, the growth inhibitory agent may be Exatecan, for example.

Accordingly, in some embodiments, the present invention provides an immunoconjugate comprising an antibody according to the invention covalently linked via a linker to Exatecan, wherein the conjugate has the following formula (IV):

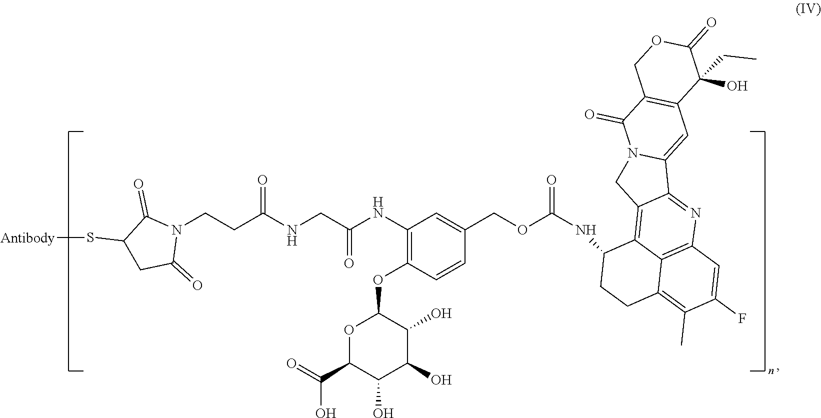

wherein S is a sulfur atom of the antibody, and wherein n is a number of [(linker)-(Exatecan)] moieties covalently linked to the antibody. The number n (also referred to as the DAR) may be e.g. between 1 and 10; in more specific embodiments, n is between 7 and 8; in even more specific embodiments, n is between 7.5 and 8.0 (i.e. about 8). In some embodiments, the antibody is mAb1.

In other embodiments, the present invention provides an immunoconjugate comprising an antibody according to the invention covalently linked via a linker to Exatecan, wherein the conjugate has the following formula (V):

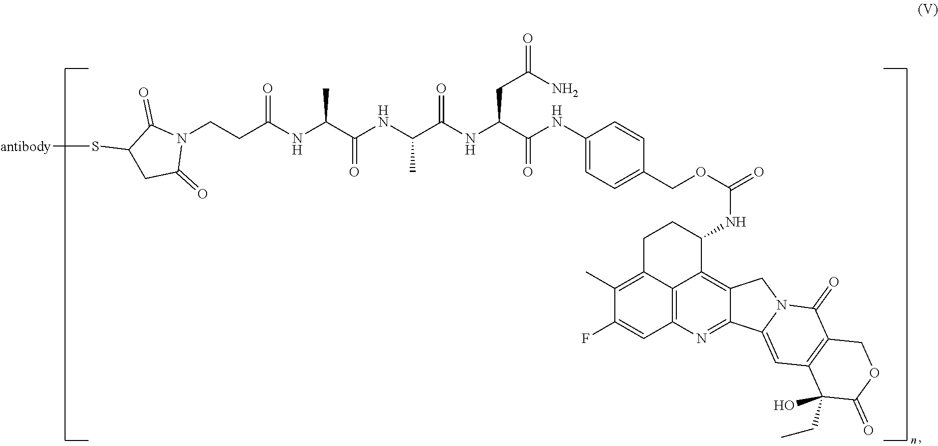

wherein S is a sulfur atom of the antibody, and wherein n is a number of [(linker)-(Exatecan)] moieties covalently linked to the antibody. The number n (also referred to as the DAR) may be e.g. between 1 and 10; in more specific embodiments, n is between 7 and 8; in even more specific embodiments, n is between 7.5 and 8.0 (i.e. about 8). In some embodiments, the antibody is mAb1.

In some embodiments, in an immunoconjugate of the invention such as the Exatecan conjugates with glucuronidase- or legumain-cleavable linkers as described above, the linker is covalently attached to the antibody at a sulfur atom of a cysteine residue of the antibody. For example, this cysteine residue of the antibody may be one of the cysteine residues capable of forming an interchain disulfide bond (also referred to herein as an interchain disulfide bridge). As there are four interchain disulfide bonds in an IgG1 antibody, involving a total of eight cysteine residues, attachment of the linker to the antibody at a sulfur atom of such cysteine residues provides that the DAR may be up to 8 and, in such cases, the DAR is typically between 7 and 8, such as between 7.5 and 8.0 (i.e. about 8), provided that the antibody is an IgG1 or has the same number of interchain disulfide bonds as an IgG1.

Accordingly, in some embodiments, the present invention provides an immunoconjugate comprising an antibody according to the invention covalently linked via a linker to Exatecan, wherein the conjugate has the following formula (VI):

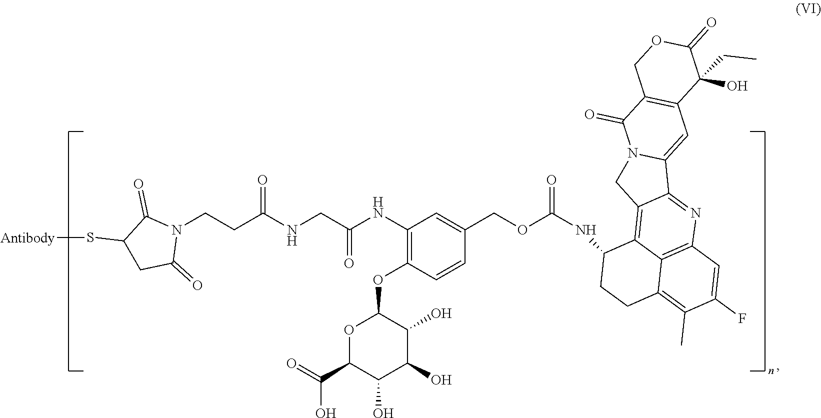

wherein S is a sulfur atom of a cysteine of the antibody, and wherein n is a number of [(linker)-(Exatecan)] moieties covalently linked to the antibody. The number n (also referred to as the DAR) may be e.g. between 1 and 10; in more specific embodiments, n is between 7 and 8; in even more specific embodiments, n is between 7.5 and 8.0 (i.e. about 8).

In other embodiments, the present invention provides an immunoconjugate comprising an antibody according to the invention covalently linked via a linker to Exatecan, wherein the conjugate has the following formula (VII):

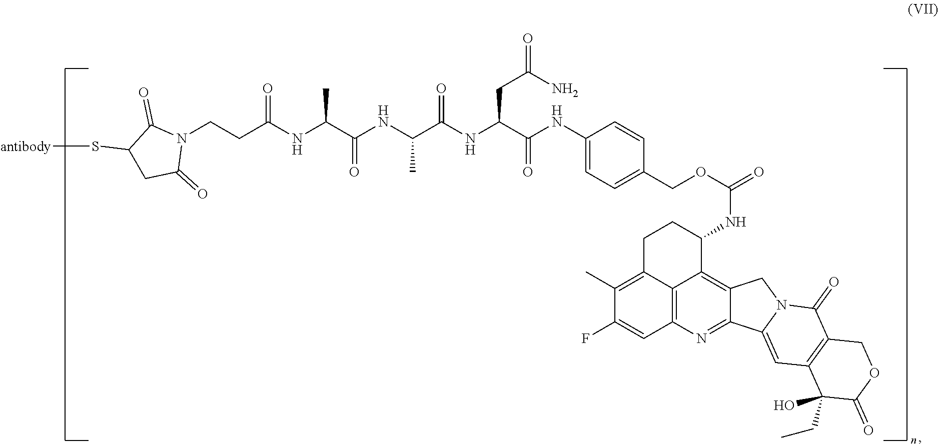

wherein S is a sulfur atom of a cysteine of the antibody, and wherein n is a number of [(linker)-(Exatecan)] moieties covalently linked to the antibody. The number n (also referred to as the DAR) may be e.g. between 1 and 10; in more specific embodiments, n is between 7 and 8; in even more specific embodiments, n is between 7.5 and 8.0 (i.e. about 8).

In any of the immunoconjugates described above, any antibody of the invention (as described herein above and below) may be used. In some embodiments, the immunoconjugate of the invention comprises mAb1 as the antibody.

Accordingly, in some embodiments, the present invention provides an immunoconjugate comprising mAb1 covalently linked via a linker to Exatecan, wherein the conjugate has the following formula (VIII):

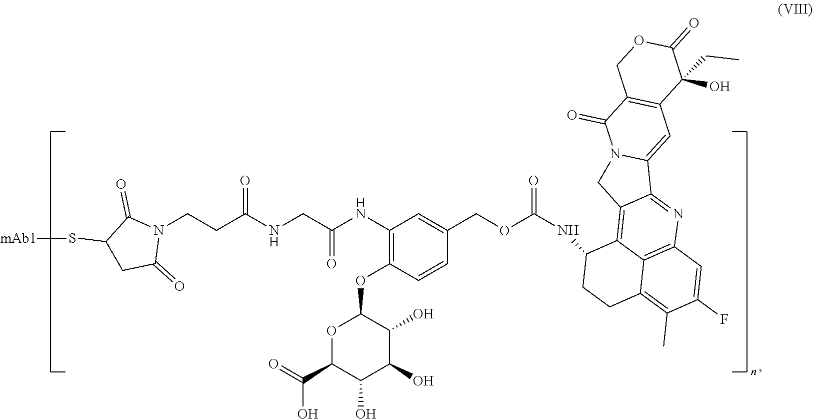

wherein S is a sulfur atom of a cysteine of the antibody mAb1, and wherein n is a number of [(linker)-(Exatecan)] moieties covalently linked to mAb1. The number n (also referred to as the DAR) may be e.g. between 1 and 10; in more specific embodiments, n is between 7 and 8; in even more specific embodiments, n is between 7.5 and 8.0 (i.e. about 8). In some embodiments, S is a sulfur atom of a cysteine of mAb1 capable of forming an interchain disulfide bridge and the DAR is about 8. An example of such an immunoconjugate (namely “ADC1”) is further described in the Examples.

In other embodiments, the present invention provides an immunoconjugate comprising mAb1 covalently linked via a linker to Exatecan, wherein the conjugate has the following formula (IX):

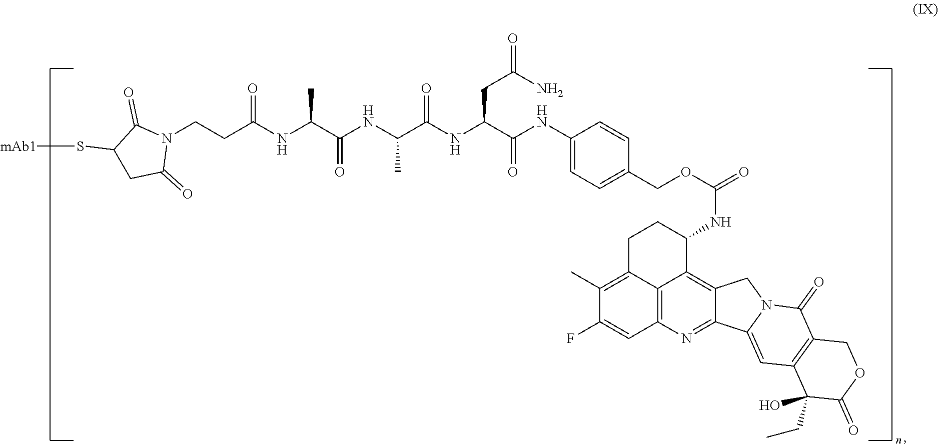

wherein S is a sulfur atom of a cysteine of the antibody mAb1, and wherein n is a number of [(linker)-(Exatecan)] moieties covalently linked to mAb1. The number n (also referred to as the DAR) may be e.g. between 1 and 10; in more specific embodiments, n is between 7 and 8; in even more specific embodiments, n is between 7.5 and 8.0 (i.e. about 8). In some embodiments, S is a sulfur atom of a cysteine of mAb1 capable of forming an interchain disulfide bridge and the DAR is about 8. The number of cytotoxic and/or cytostatic agents linked to the antigen binding moiety of an anti-huLRRC15 ADC can vary (called the “drug-to-antibody ratio,” or “DAR”), and will be limited only by the number of available attachments sites on the antigen binding moiety and the number of agents linked to a single linker. As long as the anti-GD2 ADC does not exhibit unacceptable levels of aggregation under the conditions of use and/or storage, anti-GD2 ADCs with DARs of twenty, or even higher, are contemplated.

In other embodiments of the present invention, the linker may be a “non-cleavable linker” (for example an SMCC linker). Release of the growth inhibitory agent from the antibody can occur upon lysosomal degradation of the antibody.

In other embodiments of the invention, the immunoconjugate may be a fusion protein comprising an antibody of the invention and a cytotoxic or growth inhibitory polypeptide (as the growth inhibitory agent); such fusion proteins may be made by recombinant techniques or by peptide synthesis, i.e, methods well known in the art. A molecule of encoding DNA may comprise respective regions encoding the two portions of the conjugate (antibody and cytotoxic or growth inhibitory polypeptide, respectively) either adjacent to one another or separated by a region encoding a linker peptide.

In some embodiments of the present invention, while keeping adequate ADCC activity to attack tumor cells, pain was reduced by avoiding CDC via Fc point mutation (K322A) in the hu14.18 Ab as supported by experimental data from the rat allodynia model indicating a partial alleviation of mechanical allodynia (Slart et al., 1997, Sorkin et al., 2010).

To this end, an ADC (Molecule 1) was designed by conjugating the humanized ch14.18-derived Ab with Exatecan, a potent DNA topoisomerase I inhibitor, to induce tumor cell apoptosis after ADC internalization and intracellular enzymatic release of the payload in lysosomes. Moreover, both ADCC and CDC activity were engineered out from the Fc part of the Ab to attenuate nerve injury by these mechanisms. Since topoisomerase I inhibitors do not induce peripheral neuropathy by themselves (Verschraegen et al., 2000, Rowinsky 2005) or as part of an ADC (Ogitani et al., 2016), in contrast to microtubule inhibitors (Stagg et al., 2016), the cell killing of relatively high GD2-expressing tumors, by the payload, was achieved while sparing the peripheral nervous system (PNS) having low GD2-expression, thereby, mitigating pain effects.

The antibodies of the present invention may also be used in directed enzyme prodrug therapy such as antibody-directed enzyme prodrug therapy by conjugating the antibodies to a prodrug-activating enzyme which converts a prodrug (e.g. a peptidyl chemotherapeutic agent, see WO81/01145) to an active cytotoxic drug (see, for example, WO 88/07378 and U.S. Pat. No. 4,975,278). The enzyme component of an immunoconjugate useful for ADEPT may include any enzyme capable of acting on a prodrug in such a way as to convert it into its more active, cytotoxic form. Enzymes that are useful in this context include, but are not limited to, alkaline phosphatase useful for converting phosphate-containing prodrugs into free drugs; arylsulfatase useful for converting sulfate-containing prodrugs into free drugs; cytosine deaminase useful for converting non-toxic fluorocytosine into the anticancer drug 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as O-galactosidase and neuraminidase useful for converting glycosylated prodrugs into free drugs; P-lactamase useful for converting drugs derivatized with P-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. The enzymes can be covalently bound to the antibodies of the invention by techniques well known in the art, such as the use of the linkers discussed above.

Suitable methods for preparing an immunoconjugate of the invention are well known in the art (see e.g. Hermanson G. T., Bioconjugate Techniques, Third Edition, 2013, Academic Press). For instance, methods of conjugating a cytotoxic drug to an antibody via a linker that attaches covalently to cysteine residues of interchain disulfide bridges of the antibody are well known.

In general, an immunoconjugate of the present invention can be obtained e.g. by a process comprising the steps of:
(i) preparing a compound comprising the linker and the growth inhibitory agent (e.g. cytotoxic drug), also referred to herein as a “drug-linker compound”;
(ii) bringing into contact an optionally buffered aqueous solution of an antibody according to the invention with a solution of the drug-linker compound;
(iii) then optionally separating the conjugate which was formed in (ii) from the unreacted antibody and/or drug-linker compound.

The aqueous solution of antibody can be buffered with buffers such as e.g. histidine, potassium phosphate, acetate, citrate or N-2-Hydroxyethylpiperazine-N′-2-ethanesulfonic acid (Hepes buffer). The buffer may be chosen depending upon the nature of the antibody. The drug-linker compound can be dissolved e.g. in an organic polar solvent such as dimethyl sulfoxide (DMSO) or dimethylacetamide (DMA).

For conjugation to the cysteine residues of an antibody, the antibody is subjected to reduction (e.g. using TCEP) before step (ii). Suitable reduction conditions to reduce only the interchain disulfide bonds are known in the art.

The reaction temperature for conjugation is usually between 2° and 40° C. The reaction time can vary and is typically from 1 to 24 hours. The reaction between the antibody and the drug-linker compound can be monitored by size exclusion chromatography (SEC) with a refractometric and/or UV detector. If the conjugate yield is too low, the reaction time can be extended.

A number of different chromatography methods can be used by the person skilled in the art in order to perform the separation of step (iii): the conjugate can be purified e.g. by SEC, adsorption chromatography (such as ion exchange chromatography, IEC), hydrophobic interaction chromatography (HIC), affinity chromatography, mixed-support chromatography such as hydroxyapatite chromatography, or high performance liquid chromatography (HPLC) such as reverse-phase HPLC. Purification by dialysis or filtration or diafiltration can also be used.

After step (ii) and/or (iii), the conjugate-containing solution can be subjected to an additional step (iv) of purification e.g. by chromatography, ultrafiltration and/or diafiltration. Such an additional step of purification e.g. by chromatography, ultrafiltration and/or diafiltration can also be performed with the antibody-containing solution after the reduction reaction, in cases where reduction is performed prior to conjugation.

The conjugate is recovered at the end of such a process in an aqueous solution. The drug-to-antibody ratio (DAR) is a number that can vary with the nature of the antibody and of the drug-linker compound used along with the experimental conditions used for the conjugation (such as the ratio (drug-linker compound)/(antibody), the reaction time, the nature of the solvent and of the cosolvent if any). Thus, the contact between the antibody and the drug-linker compound can lead to a mixture comprising several conjugates differing from one another by different drug-to-antibody ratios. The DAR that is determined is thus an average value.

Performing conjugation at the cysteine residues of interchain disulfide bridges using an antibody that has four interchain disulfide bridges (e.g. mAb1 or any IgG1 antibody)—which is a method well known in the art-offers the advantage that a relatively homogeneous DAR of about 8 can be achieved by choosing reaction conditions that allow conjugation to proceed to completion (or at least close to completion).

An exemplary method which can be used to determine the DAR consists of measuring spectrophotometrically the ratio of the absorbance at of a solution of purified conjugate at λ_{D} and 280 nm. 280 nm is a wavelength generally used for measuring protein concentration, such as antibody concentration. The wavelength λ_{D} is selected so as to allow discriminating the drug from the antibody, i.e. as readily known to the skilled person, λ_{D} is a wavelength at which the drug has a high absorbance and λ_{D} is sufficiently remote from 280 nm to avoid substantial overlap in the absorbance peaks of the drug and antibody. For instance, λ_{D} may be selected as being 370 nm for Exatecan (or for camptothecin or other camptothecin analogs), or 252 nm for maytansinoid molecules.

A method of DAR calculation may be derived e.g. from Antony S. Dimitrov (ed), LLC, 2009, Therapeutic Antibodies and Protocols, vol 525, 445, Springer Science: The absorbances for the conjugate at λ_{D} (A_{λD}) and at 280 nm (A₂₈₀) are measured either on the monomeric peak of the size exclusion chromatography (SEC) analysis (allowing to calculate the “DAR (SEC)” parameter) or using a classic spectrophotometer apparatus (allowing to calculate the “DAR (UV)” parameter). The absorbances can be expressed as follows:

$${A}_{λ D} = ( {C}_{D} \times {ε}_{D λ D} ) + ( {C}_{A} \times {ε}_{A λ D} )$$

$${A}_{280} = ( {C}_{D} \times {ε}_{D 280} ) + ( {C}_{A} \times {ε}_{A 280} )$$

wherein:
C_{D} and C_{A} are respectively the concentrations in the solution of the drug and of the antibody
ε_{DλD} and ε_{D280} are respectively the molar extinction coefficients of the drug at λ_{D} and 280 nm
ε_{AλD} and ε_{A280} are respectively the molar extinction coefficients of the antibody at λ_{D} and 280 nm.

Resolution of these two equations with two unknowns leads to the following equations:

$${C}_{D} = [ ( {ε}_{A 280} \times {A}_{λ D} ) - ( {ε}_{A λ D} \times {A}_{280} ) ] / [ ( {ε}_{D λ D} \times {ε}_{A 280} ) - ( {ε}_{A λ D} \times {ε}_{D 280} ) ]$$

$${C}_{A} = [ {A}_{280} - ( {C}_{D} \times {ε}_{D 280} ) ] / {ε}_{A 280}$$

The average DAR is then calculated from the ratio of the drug concentration to that of the antibody: DAR=C_{D}/C_{A}.
Exemplary methods for preparing an immunoconjugate of the invention are described in the Examples.

### Drug-Linker Compounds

The present invention also provides compounds comprising a linker and a growth inhibitory agent (e.g. a cytotoxic drug), also referred to herein as “drug-linker compounds”. For instance, the present invention provides a compound of the following formula (X):

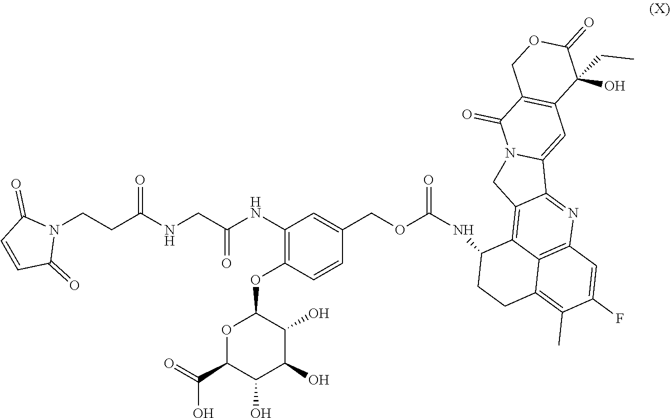 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CNC(=O)CCN2C(=O)C=CC2=O)c1)CC3]

or a physiologically acceptable salt thereof; this compound is also referred to herein as “drug-linker compound 1”, “compound DL1” or “DL1”.

The present invention also provides a compound of the following formula (XI):

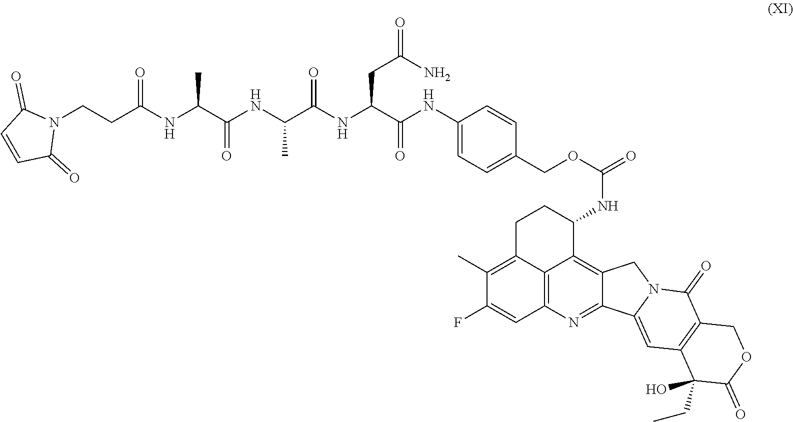 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CC(N)=O)NC(=O)[C@H](C)NC(=O)[C@H](C)NC(=O)CCN2C(=O)C=CC2=O)cc1)CC3]

or a physiologically acceptable salt thereof; this compound is also referred to herein as “drug-linker compound 2”, “compound DL2” or “DL2”.

These drug-linker compounds may be used to prepare immunoconjugates of the invention as described herein above and below.

The drug-linker compounds of the invention (e.g. those of formula (X) or (XI) depicted above) may be prepared by chemical synthesis, for instance as described in the Examples further below.

### Pharmaceutical Compositions

The antibodies or immunoconjugates of the invention may be combined with pharmaceutically acceptable carriers, diluents and/or excipients, and optionally with sustained-release matrices including but not limited to the classes of biodegradable polymers, non-biodegradable polymers, lipids or sugars, to form pharmaceutical compositions.

Thus, another aspect of the invention relates to a pharmaceutical composition comprising an antibody or an immunoconjugate of the invention and a pharmaceutically acceptable carrier, diluent and/or excipient.

“Pharmaceutical” or “pharmaceutically acceptable” refers to molecular entities and compositions that do not produce an adverse, allergic or other unwanted reaction when administered to a mammal, especially a human, as appropriate. A pharmaceutically acceptable carrier, diluent or excipient refers to a non-toxic solid, semi-solid or liquid filler, diluent, encapsulating material or formulation auxiliary of any type.

As used herein, “pharmaceutically acceptable carriers” include any and all solvents, dispersion media, coatings, antibacterial and antifungal agents, and the like that are physiologically compatible. Examples of suitable carriers, diluents and/or excipients include, but are not limited to, one or more of water, amino acids, saline, phosphate buffered saline, buffer phosphate, acetate, citrate, succinate; amino acids and derivates such as histidine, arginine, glycine, proline, glycylglycine; inorganic salts such as NaCl or calcium chloride; sugars or polyalcohols such as dextrose, glycerol, ethanol, sucrose, trehalose, mannitol; surfactants such as polysorbate 80, polysorbate 20, poloxamer 188; and the like, as well as combination thereof. In many cases, it will be useful to include isotonic agents, such as sugars, polyalcohols, or sodium chloride in a pharmaceutical composition, and the formulation may also contain an antioxidant such as tryptamine and/or a stabilizing agent such as Tween 20.

The form of the pharmaceutical compositions, the route of administration, the dosage and the regimen naturally depend upon the condition to be treated, the severity of the illness, the age, weight, and gender of the patient, etc.

The pharmaceutical compositions of the invention can be formulated for a topical, oral, parenteral, intranasal, intravenous, intramuscular, subcutaneous or intraocular administration and the like.

In an embodiment, the pharmaceutical compositions contain vehicles which are pharmaceutically acceptable for a formulation for injection. These may be isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical composition can be administrated through drug combination devices.

The doses used for the administration can be adapted as a function of various parameters, and for instance as a function of the mode of administration used, of the relevant pathology, or alternatively of the desired duration of treatment.

To prepare pharmaceutical compositions, an effective amount of the antibody or immunoconjugate of the invention may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions; in all such cases, the form must be sterile and injectable with the appropriate device or system for delivery without degradation, and it must be stable under the conditions of manufacture and storage and must be preserved against the contaminating action of microorganisms, such as bacteria and fungi.

Solutions of active compounds as free base or pharmacologically acceptable salts can be prepared in water suitably mixed with a surfactant. Dispersions can also be prepared in glycerol, liquid polyethylene glycols, and mixtures thereof and in oils. Under ordinary conditions of storage and use, these preparations may contain a preservative to prevent the growth of microorganisms.

An antibody or immunoconjugate of the invention can be formulated into a pharmaceutical composition in a neutral or salt form. Pharmaceutically acceptable salts include the acid addition salts (formed with the free amino groups of the protein) which are formed with inorganic acids such as, for example, hydrochloric or phosphoric acids, or such organic acids as acetic, oxalic, tartaric, or mandelic acid, and the like. Salts formed with the free carboxyl groups can also be derived from inorganic bases such as, for example, sodium, potassium, ammonium, calcium, or ferric hydroxides, and such organic bases as isopropylamine, trimethylamine, glycine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In some cases, it may be desirable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions can be prepared by incorporating the active compounds in the required amount in the appropriate solvent with any of the other ingredients enumerated above, as required, followed by filtered sterilization. Generally, dispersions can be prepared by incorporating the various sterilized active ingredients into a sterile vehicle which contains the basic dispersion medium and the required other ingredients from those enumerated above. In the case of sterile powders for the preparation of sterile injectable solutions, methods of preparation include vacuum-drying and freeze-drying techniques which yield a powder of the active ingredient plus any additional desired ingredient from a previously sterile-filtered solution thereof.

The preparation of more concentrated, or highly concentrated solutions for direct injection is also contemplated, where the use of DMSO as solvent is envisioned to result in extremely rapid penetration, delivering high concentrations of the active agents to a small tumor area.

Upon formulation, solutions can be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution can be suitably buffered if necessary and the liquid diluent first rendered isotonic with sufficient saline or glucose. These aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The antibody or immunoconjugate of the invention may be formulated within a therapeutic mixture to comprise e.g. about 0.01 to 100 milligrams per dose or so.

In addition to the antibody or immunoconjugate formulated for parenteral administration, such as intravenous or intramuscular injection, other pharmaceutically acceptable forms include e.g. tablets or other solids for oral administration, time release capsules, and any other form currently used.

In some embodiments, the use of liposomes and/or nanoparticles is contemplated for the introduction of polypeptides into host cells. The formation and use of liposomes and/or nanoparticles are known to those of skill in the art.

Nanocapsules can generally entrap compounds in a stable and reproducible way. To avoid side effects due to intracellular polymeric overloading, such ultrafine particles (sized around 0.1 μm) are generally designed using polymers able to be degraded in vivo. Biodegradable polyalkyl-cyanoacrylate nanoparticles, or biodegradable polylactide or polylactide coglycolide nanoparticles that meet these requirements are contemplated for use in the present invention, and such particles may be easily made by those of skill in the art.

Liposomes can be formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 A, containing an aqueous solution in the core. The physical characteristics of liposomes depend on pH, ionic strength and the presence of divalent cations.

Besides the above-mentioned examples, further pharmaceutical forms such as nanoparticles, microparticles and -capsules, implants (e.g. lipid implants), or self-solidifying or -emulsifying systems, are also contemplated.

### Therapeutic Methods and Uses

The inventors have found that an antibody of the invention (e.g. mAb1) is able to internalize as part of the GD2-antibody complex after binding. Furthermore, the target-mediated uptake of such an antibody, conjugated to a cytotoxic drug (in a preferred embodiment, Exatecan) was shown to translate into an increased in vitro cytotoxic potency in GD2-expressing tumor cells in comparison to the respective control ADC or target-negative cells. The inventors have also shown that these immunoconjugates of the invention induce a marked anti-tumor activity in vivo when used at doses range from 0.25 mg/kg to 10 mg/kg, with a single injection or multiple injections. In fact, the immunoconjugates of the invention show broad activity in a large set of in vitro and in vivo models derived from different tumor types. Moreover, immunoconjugates of the present invention were well tolerated in a non-human primate dose-range finding study. These preclinical data indicate a good therapeutic window for later clinical testing. The antibodies, immunoconjugates and pharmaceutical compositions of the invention are, therefore, useful for treating cancers that expressing GD2.

Accordingly, the present invention provides the antibody, immunoconjugate or pharmaceutical composition of the invention for use as a medicament. For instance, the invention provides the antibody, immunoconjugate or pharmaceutical composition of the invention for use in the treatment of cancer. The invention further provides a method of treating cancer, comprising administering the antibody, immunoconjugate or pharmaceutical composition of the invention to a subject in need thereof.

The cancers to be treated with antibodies, immunoconjugates, or pharmaceutical compositions of the invention is preferably a cancer expressing GD2, more preferably a cancer overexpressing GD2 as compared to normal (i.e. non-tumoral) cells of the same tissue origin. Expression of GD2 by cells may be readily assayed for instance by using an antibody according to the invention (or a commercially available anti-GD2 antibody), for instance as described in the following section “Diagnostic Uses”, and e.g. by an immunohistochemical method.

GD2 is implicated in tumor development and malignant phenotypes through enhanced cell proliferation, motility, migration, adhesion, and invasion, depending on the tumor type. This provides a rationale for targeting disialoganglioside GD2 in cancer therapy for any tumor type which overexpresses GD2. While it is not intended the present invention be limited to any specific mechanism of action, it is known anti-GD2 monoclonal antibodies target GD2-expressing tumor cells, leading to phagocytosis and destruction by means of antibody-dependent cell-mediated cytotoxicity, lysis by complement-dependent cytotoxicity, and apoptosis and necrosis through direct induction of cell death. In addition, anti-GD2 monoclonal antibodies may also prevent homing and adhesion of circulating malignant cells to the extracellular matrix.

In some embodiments, the cancers to be treated with antibodies, immunoconjugates, or pharmaceutical compositions of the present invention are neuroblastomas, melanomas, retinoblastomas, Ewing sarcomas, small cell lung cancer, breast cancer, gliomas, osteosarcomas, and soft tissue sarcomas. In selected embodiments the cancers to be treated with antibodies, immunoconjugates, or pharmaceutical compositions of the present invention are neuroblastoma and osteosarcoma.

The antibodies or immunoconjugates of the invention may be used in cancer therapy alone or in combination with any suitable growth inhibitory agent.

The antibodies of the invention may be conjugated (linked) to a growth inhibitory agent, as described above. Antibodies of the invention may thus be useful for targeting said growth inhibitory agent to cancerous cells expressing or over-expressing GD2 on their surface.

It is also well known that therapeutic monoclonal antibodies can lead to the depletion of cells bearing the antigen specifically recognized by the antibody. This depletion can be mediated through at least three mechanisms: antibody mediated cellular cytotoxicity (ADCC), complement dependent lysis, and direct inhibition of tumor growth through signals mediated by the antigen targeted by the antibody.

“Antibody-dependent cell-mediated cytotoxicity” or “ADCC” refers to a form of cytotoxicity in which antibodies bound to Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell. To assess ADCC activity of a molecule of interest, an in vitro ADCC assay, such as that described in U.S. Pat. No. 5,500,362 or 5,821,337 may be performed.

“Complement dependent cytotoxicity” or “CDC” refers to the lysis of a target cell in the presence of complement. Activation of the classical complement pathway is initiated by the binding of the first component of the complement system to antibodies which are bound to their cognate antigen. To assess complement activation, a CDC assay, e.g. as described in Gazzano-Santoro et al. (Journal of Immunological Methods. 1997 March; 202 (2): 163-171) may be performed.

In some embodiments, an antibody of the invention may be an antibody with a modified amino acid sequence that results in reduced or eliminated binding to most Fcγ receptors, which can reduce uptake and toxicity in normal cells and tissues expressing such receptors, e.g. macrophages, liver sinusoidal cells etc.,

An aspect of the invention relates to a method of treating cancer, comprising administering a therapeutically effective amount of the antibody, immunoconjugate or pharmaceutical composition of the invention to a subject in need thereof.

In the context of the invention, the term “treating” or “treatment”, as used herein, means reversing, alleviating, inhibiting the progress of, or preventing the disorder or condition to which such term applies, or one or more symptoms of such disorder or condition. By the term “treating cancer” as used herein is meant the inhibition of the growth of malignant cells of a tumor and/or the progression of metastases from said tumor. Such treatment can also lead to the regression of tumor growth, i.e., the decrease in size of a measurable tumor. For instance, such treatment can lead to the complete regression of the tumor or metastasis.

In the context of the therapeutic applications of the present invention, the term “subject” or “patient” or “subject in need thereof” or “patient in need thereof” refers to a subject (e.g. a human or non-human mammal) affected or likely to be affected by a tumor. For instance, said patient may be a patient who has been determined to be susceptible to a therapeutic agent targeting GD2, in particular to an antibody or immunoconjugate according to the invention, for instance according to a method as described herein below.

By a “therapeutically effective amount” is meant a sufficient amount to treat said cancer disease at a reasonable benefit/risk ratio applicable to any medical treatment. It will be understood, however, that the total daily usage of the antibodies, immunoconjugates and pharmaceutical compositions (collectively referred to as the “therapeutic agent”) of the present invention will be decided by the attending physician within the scope of sound medical judgment. The specific therapeutically effective dose level for any particular patient will depend upon a variety of factors including the disorder being treated and the severity of the disorder; activity of the specific therapeutic agent employed; the age, body weight, general health, sex and diet of the patient; the time of administration, route of administration, and rate of excretion of the specific therapeutic agent employed; the duration of the treatment; drugs used in combination or coincidental with the specific therapeutic agent employed; and like factors well known in the medical arts. For example, it is well known within the skill of the art to start doses of the compound at levels lower than those required to achieve the desired therapeutic effect and to gradually increase the dosage until the desired effect is achieved.

The antibody, immunoconjugate or pharmaceutical composition of the invention may also be used for inhibiting the progression of metastases of a cancer.

Antibodies, immunoconjugates or pharmaceutical compositions of the invention may also be used in combination with any other therapeutic intervention for treating a cancer (e.g. adjuvant therapy) and/or for reducing the growth of a metastatic cancer. For instance, the other therapeutic intervention for such combination may be a standard-of-care (SOC) therapeutic agent for the cancer to be treated.

Efficacy of the treatment with an antibody or immunoconjugate or pharmaceutical composition according to the invention may be readily assayed in vivo, for instance in a mouse model of cancer and by measuring e.g. changes in tumor volume between treated and control groups, % tumor regression, partial regression or complete regression.

### Diagnostic Uses

GD2 has been reported to be highly expressed on the surface of cancer cells such as neuroblastomas, melanomas, retinoblastomas, Ewing sarcomas, small cell lung cancer, breast cancer, gliomas, osteosarcomas, soft tissue sarcomas or other solid tumors expressing GD2.

Therefore, GD2 may be considered a cancer marker and has the potential to be used to indicate the effectiveness of an anti-cancer therapy or to detect recurrence of the disease.

In an embodiment, the antibodies of the invention can be used as component of an assay in the context of a therapy targeting GD2 expressing tumors, in order to determine susceptibility of the patient to the therapeutic agent, monitor the effectiveness of the anti-cancer therapy or detect recurrence of the disease after treatment. In some embodiments, the same antibody of the invention can be used both as component of the therapeutic agent and as component of the diagnostic assay.

Thus, a further aspect of the invention relates to a use of an antibody according to the invention for detecting GD2 expression ex vivo in a biological sample from a subject. Another aspect of the invention relates to the use of an antibody of the invention for detecting GD2 expression in vivo in a subject. When used for detection of GD2, the antibody may be labelled with a detectable molecule such as e.g. a fluorophore or an enzyme.

Detection of GD2 may be intended for:
a) diagnosing the presence of a cancer in a subject, or
b) determining susceptibility of a patient having cancer to a therapeutic agent targeting GD2, in particular an antibody or immunoconjugate according to the invention, or
c) monitoring effectiveness of an anti-GD2 cancer therapy or detecting a cancer relapse after anti-GD2 cancer therapy, in particular wherein said therapy is therapy with an antibody or immunoconjugate according to the invention;
   by detecting expression of the surface GD2 on tumor cells.

In some embodiments, the antibodies of the present invention are intended for an in vitro or ex vivo diagnostic use. For example, GD2 may be detected using an antibody of the invention in vitro or ex vivo in a biological sample obtained from a subject. Use according to the invention may also be an in vivo use. For example, an antibody according to the invention can be administered to the subject and antibody-cell complexes can be detected and/or quantified, whereby the detection of said complexes is indicative of a cancer.

The invention further relates to an in vitro or ex vivo method of detecting the presence of a cancer in a subject, comprising the steps of:
(a) contacting a biological sample from a subject with an antibody according to the invention, in particular in conditions suitable for the antibody to form complexes with said biological sample;
(b) measuring the level of antibody bound to said biological sample; and
(c) detecting the presence of a cancer by comparing the measured level of bound antibody with a control, an increased level of bound antibody compared to control being indicative of a cancer.

The invention also relates to an in vitro or ex vivo method of determining susceptibility of a patient having cancer to a therapeutic agent targeting GD2, in particular an antibody or immunoconjugate according to the invention, which method comprises the steps of:
(a) contacting a biological sample from a patient having cancer with an antibody according to the invention, in particular in conditions suitable for the antibody to form complexes with said biological sample;
(b) measuring the level of antibody bound to said biological sample; and
(c) comparing the measured level of bound antibody to said biological sample with the level of antibody bound to a control;
   wherein an increased level of bound antibody to said biological sample compared to control is indicative of a patient susceptible to a therapeutic agent targeting GD2.

In the above methods, said control can be a normal, non-cancerous biological sample of the same type, or a reference value determined as representative of the antibody binding level in a normal biological sample of the same type.

In an embodiment, the antibodies of the invention are useful for diagnosing a GD2 expressing cancer, such as a colorectal cancer, gastric cancer, non-small cell lung cancer, pancreatic cancer, esophageal cancer, prostate cancer or other solid tumors expressing GD2.

The invention further relates to an in vitro or ex vivo method of monitoring effectiveness of anti-GD2 cancer therapy, comprising the steps of:
(a) contacting a biological sample from a subject undergoing anti-GD2 cancer therapy with an antibody according to the invention, in particular in conditions suitable for the antibody to form complexes with said biological sample;
(b) measuring the level of antibody bound to said biological sample; and
(c) comparing the measured level of bound antibody with the level of antibody bound to a control;
   wherein a decreased level of bound antibody to said biological sample compared to control is indicative of effectiveness of said anti-GD2 cancer therapy. In said method, an increased level of bound antibody to said biological sample compared to control would be indicative of ineffectiveness of said anti-GD2 cancer therapy. In an embodiment of this method of monitoring effectiveness, said control is a biological sample of the same type as the biological sample submitted to analysis, but which was obtained from the subject at an earlier time point during the course of the anti-GD2 cancer therapy.

The invention further relates to an in vitro or ex vivo method of detecting cancer relapse after anti-GD2cancer therapy, comprising the steps of:
(a) contacting a biological sample from a subject, the subject having completed anti-GD2cancer therapy, with an antibody according to the invention, in particular in conditions suitable for the antibody to form complexes with said biological sample;
(b) measuring the level of antibody bound to said biological sample; and
(c) comparing the measured level of bound antibody with the level of antibody bound to a control;
   wherein an increased level of bound antibody to said biological sample compared to control is indicative of cancer relapse after anti-GD2 cancer therapy. Said control may be, in particular, a biological sample of the same type as the biological sample submitted to analysis, but which was obtained from the subject previously, namely upon or after completion of the anti-GD2 cancer therapy.

Said anti-GD2 cancer therapy is a therapy using an antibody or immunoconjugate according to the invention. Said anti-GD2 cancer therapy targets a GD2 expressing cancer, such as neuroblastomas, melanomas, retinoblastomas, Ewing sarcomas, small cell lung cancer, breast cancer, gliomas, osteosarcomas, and soft tissue sarcomas, or other solid tumors expressing GD2.

In some embodiments, antibodies of the invention may be labelled with a detectable molecule or substance, such as a fluorescent molecule or fluorophore, a radioactive molecule, an enzyme or any other labels known in the art that provide (either directly or indirectly) a signal.

As used herein, the term “labeled”, with regard to the antibody according to the invention, is intended to encompass direct labeling of the antibody by coupling (i.e., physically linking) a detectable substance, such as a radioactive agent or a fluorophore (e.g. fluorescein isothiocyanate (FITC) or phycoerythrin (PE) or Indocyanine (Cy5)) to the polypeptide, as well as indirect labeling of the polypeptide by reactivity with a detectable substance.

An antibody of the invention may be labelled with a radioactive molecule by any method known to the art. For example, radioactive molecules include but are not limited to radioactive atoms for scintigraphic studies such as I¹²³, I¹²⁴, In¹¹¹, Re¹⁸⁶, Re¹⁸⁸, Tc⁹⁹. Antibodies of the invention may also be labelled with a spin label for nuclear magnetic resonance (NMR) imaging (also known as magnetic resonance imaging, MRI), such as iodine-123, indium-111, fluorine-19, carbon-13, nitrogen-15, oxygen-17, gadolinium, manganese or iron.

A “biological sample” encompasses a variety of sample types obtained from a subject that can be used in a diagnostic or monitoring assay. Biological samples include but are not limited to blood and other liquid samples of biological origin, solid tissue samples such as a biopsy specimen or tissue cultures or cells derived therefrom, and the progeny thereof. Therefore, biological samples encompass clinical samples, cells in culture, cell supernatants, cell lysates, serum, plasma, biological fluid, and tissue samples, such as tumor samples.

In some embodiments, the biological sample may be a formalin-fixed and paraffin-embedded (FFPE) or frozen tissue sample.

The invention also relates to an in vivo method of detecting the presence of a cancer in a subject, comprising the steps of:
a) administering an antibody according to the invention to a patient, wherein the antibody is labelled with a detectable molecule;
b) detecting localization of said antibody in the patient by imaging, e.g. by detecting the detectable molecule.

In said method, the cancer may be a GD2 expressing cancer, such as neuroblastomas, melanomas, retinoblastomas, Ewing sarcomas, small cell lung cancer, breast cancer, gliomas, osteosarcomas, and soft tissue sarcomas or other solid tumors expressing GD2.

Antibodies of the invention may also be useful for staging of cancer (e.g., in radio imaging). They may be used alone or in combination with other cancer markers.

The terms “detection” or “detected” as used herein include qualitative and/or quantitative detection (i.e. measuring levels) with or without reference to a control.

In the context of the invention, the term “diagnosing”, as used herein, means the determination of the nature of a medical condition, intended to identify a pathology which affects the subject, based on a number of collected data.

### Kits

Finally, the invention also provides kits comprising at least one antibody or immunoconjugate of the invention. Kits containing antibodies of the invention can find use in detecting the surface protein GD2, or in therapeutic or diagnostic assays. Kits of the invention can contain an antibody coupled to a solid support, e.g., a tissue culture plate or beads (e.g., sepharose beads). Kits can be provided which contain antibodies for detection and quantification of the surface protein GD2 in vitro, e.g. in an ELISA or a Western blot. Such an antibody useful for detection may be provided with a label such as a fluorescent or radiolabel.

### BRIEF DESCRIPTION OF THE SEQUENCES

### Amino Acid Sequences:

### Primary Structure of Hu14.18-IgG1.4 (K322A)-deIK Antibody:

```
Heavy chain sequence (SEQ ID NO: 4):
    1 EVQLVQSGAE VEKPGASVKI SCKASGSSFT GYNMNWVRQN IGKSLEWIGA IDPYYGGTSY
  61 NQKFKGRATL TVDKSTSTAV MHLKSLRSED TAVYYCVSGM EYWGQGTSVT VSSASTKGPS
121 VFPLAPSSKS TSGGTAALGC LVKDYEPEPV TVSWNSGALT SGVHTFPAVL QSSGLYSLSS
181 VVTVPSSSLG TQTYICNVNH KPSNTKVDKR VEPKSCDKTH TCPPCPAPPV AGPSVFLFPP
241 [symbol]  TLMISR TPEVTCVVVG VSHEDPEVKF NWVVDGVEVH NAKTKPREEQ YNSTVRVVSV
301 LTVLHQDWLN GKEVKCAVSN KALPSSIEKT ISKAKGQPRE PQVYTLPPSR EEMTKNQVSL
361 TCLVKGFYPS DIAVENESNG QPENNYKTTP PVLDSDGSFF LYSKLTVDKS RWQQGNVFSC
421 SVMHEALHNH YTQKSLSLSP G
Light chain sequence (SEQ ID NO: 1):
    1 DVVNTQTPLS LPVTPGEPAS ISCRSSQSLV HRNGNTYLHW VLQKPGQSPK LLINKVSNRF
  61 SGVPSRFSGS GSGTDFTLKI SRVEASDLGV VFCSQSTHVP PLTFGAGTKL ELKRTVAAPS
121 VFIFPPSDEQ LKSGTASVVC LLNNFYPREA KVQNKVDNAL QSGNSQESVT EQDSKDSTYS
181 LSSTLTLSKA DYEKHKVYAC EVTHQGLSSP VTKSFNRGEC
```

[symbol]

indicates data missing or illegible when filed

### Preferred Embodiments of Anti-GD2 ADCs

In a preferred embodiment, the present invention describes a new ADC drug which was fully optimized according to the following summary:

Selection of an antibody with suitable affinity, good cellular binding capacity and efficient internalization, ensuring sufficient payload disposition after antibody binding to cancer cells. The antibody hu14.18-IgG1.4 (K322A)-delK selected is based on the known CDR sequences of dinutuximab (ch14.18), but further optimized by humanization to reduce immunogenicity risks in patients.

The antibody moiety contains a HC K322A mutation which eliminates complement activation. This mutation reduces pain side effects, as demonstrated by a pilot trial of humanized anti-GD2 monoclonal antibody (hu14.18K322A) in combination with other drugs, including chemotherapy.

The antibody moiety is sequence modified (IgG1.4 format) which nearly eliminates binding to most Fcγ receptors. Preclinical tolerability studies in non-human primates (detailed below) demonstrates this modification together with the K322A mutation most is responsible for a near full reduction of pain effects and neurological tissue damages. As a prospective therapeutic, therefore, such a IgG1.4 modification in an ADC could reduce uptake and associated toxicities in other normal cells and tissues expressing Fcγ receptors (e.g., macrophages and liver sinusoidal cells)

In a preferred embodiment, the antibody moiety was conjugated to a highly potent topoisomerase-I inhibitor Exatecan to improve antitumor activity compared to naked anti-GD2 antibody therapies. In contrast to other ADC payloads such as tubulin toxins (e.g., auristatin and maytansines); this preferred ADC has lower risks of associated neurotoxicities. This is especially advantageous where the ADC target is presented on the surface of peripheral nerves.
(5) The linker connecting drug and antibody is designed to maximize systemic stability after parenteral application. Moreover the payload-linker moiety based on glucuronide-linkage system ensures good conjugatable, favourable PK characteristics and minor non-specific uptake in target-negative cells and tissues.
(6) Exatecan payload of ADCs used for this invention shows combination effects with DNA damage- and response inhibitors, e.g. ATR/ATM inhibitors, and immune checkpoint inhibitors, which might give additional benefit for patients in later clinical studies.

### EXAMPLES

### Example 1A: Synthesis of a Drug-Linker Compound with Glucuronide-Based Linker: Drug-Linker Compound 1 (DL1)

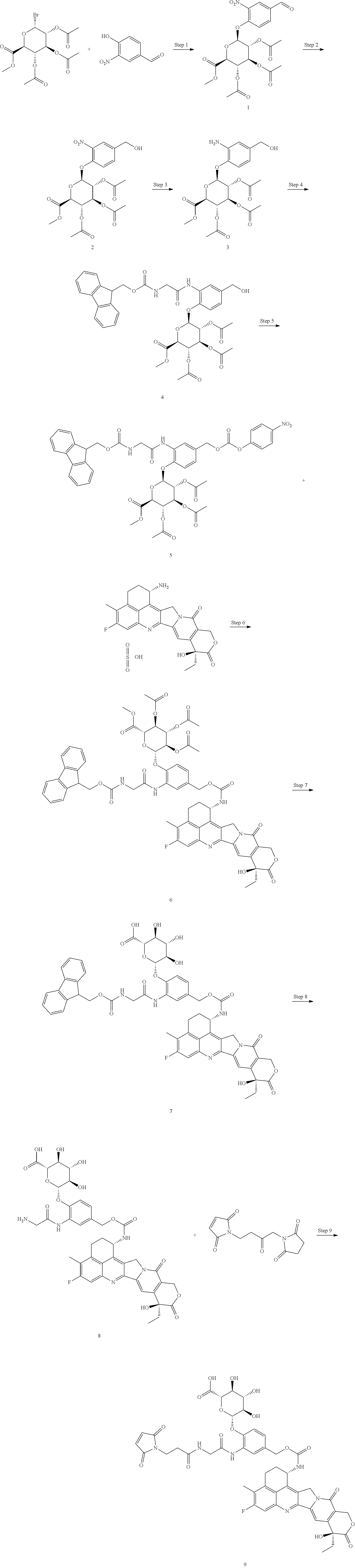

The synthetic route to compound 9 (also referred to herein as drug-linker compound 1 (DL1)).

### Protocol of Chemical Preparation

### Step 1: Compound 1

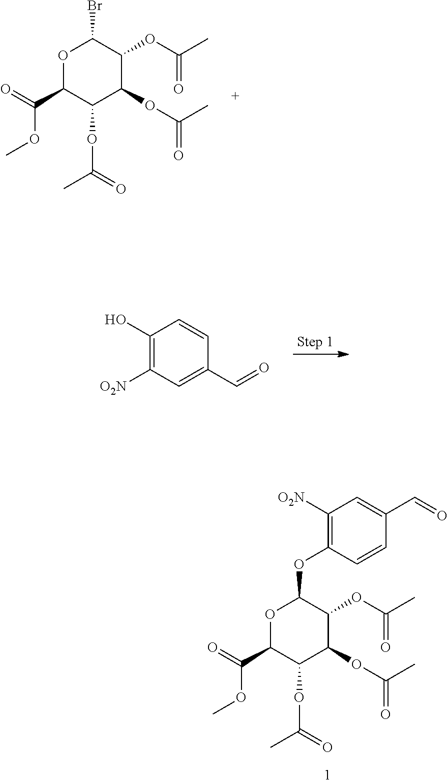 [SMILES: COC(=O)[C@H]1O[C@H](Br)[C@H](OC(C)=O)[C@@H](OC(C)=O)[C@@H]1OC(C)=O ; O=Cc1ccc(O)c([N+](=O)[O-])c1 ; COC(=O)[C@H]1O[C@@H](Oc2ccc(C=O)cc2[N+](=O)[O-])[C@H](OC(C)=O)[C@@H](OC(C)=O)[C@@H]1OC(C)=O]

To a stirred solution of (2S,3S,4S,5R,6R)-3,4,5-Triacetoxy-6-bromo-tetrahydro-pyran-2-carboxylic acid methyl ester (8.30 g; 20.90 mmol; 1.00 eq.) and 4-Hydroxy-3-nitro-benzaldehyde (5.24 g; 31.35 mmol; 1.50 eq.) in Acetonitrile (83.00 ml; 10.00 V) was added Silver (I) oxide (9.69 g; 41.80 mmol; 2.00 eq.). The reaction mixture was stirred at RT for 16 h. The reaction mixture was filtered through celite. The filtrate was concentrated under vacuum to get solid. The solid was dissolved in EtOAc and washed with 10% aqueous solution of NaHCO₃ to remove excess 4-Hydroxy-3-nitro-benzaldehyde. The organic layer was concentrated under vacuum to get compound 1 as sand color solid.

Yield: 9.0 g

Percentage Yield: 89.1%

### Analytical Data:

NMR: ¹H-NMR (400 MHZ, DMSO-d6): 9.98 (s, 1H), 8.46 (s, 1H), 8.25-8.21 (m, 1H), 7.64 (d, J=11.60 Hz, 1H), 5.94 (d, J=10.00 Hz, 1H), 5.51-5.44 (m, 1H), 5.20-5.09 (m, 2H), 4.80 (d, J=13.20 Hz, 1H), 3.64 (s, 3H), 2.09 (s, 9H).

### Step 2: Compound 2

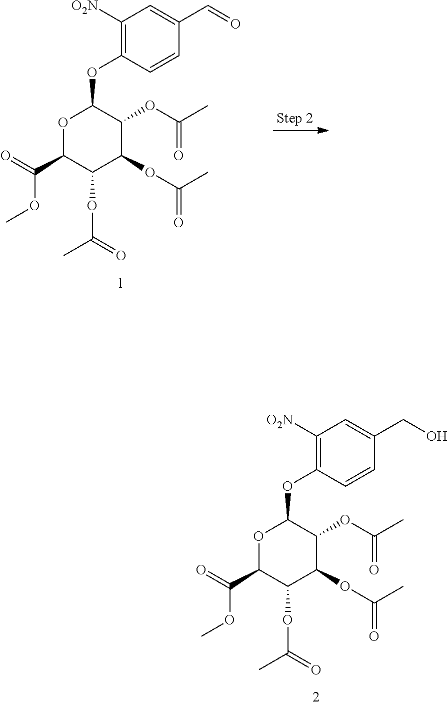 [SMILES: COC(=O)[C@H]1O[C@@H](Oc2ccc(C=O)cc2[N+](=O)[O-])[C@H](OC(C)=O)[C@@H](OC(C)=O)[C@@H]1OC(C)=O ; COC(=O)[C@H]1O[C@@H](Oc2ccc(CO)cc2[N+](=O)[O-])[C@H](OC(C)=O)[C@@H](OC(C)=O)[C@@H]1OC(C)=O]

To a stirred solution of compound 1 (9.00 g; 18.62 mmol; 1.00 eq.) in Propan-2-ol (33.00 ml; 3.67 V) and CHCl₃ (167.00 ml; 18.56 V) were added silica gel 60-120 (3.60 g; 112.09 mmol; 6.02 eq.) followed by sodium borohydride (1.80 g; 46.55 mmol; 2.50 eq.). The reaction mixture was stirred for 1 h at RT. After completion, the reaction mixture was quenched with cooled H₂O and filtered through celite. The filtrate was extracted with Dichloromethane and dried over Na₂SO₄. The solvent was concentrated to get compound 2 as off-white powder.

Yield: 8.70 g

Percentage Yield: 92.4%

### Analytical Data

LCMS: Column: ATLANTIS dC18 (50×4.6 mm) 5 μm; Mobile phase A: 0.1% HCOOH in H2O:ACN (95:5); B: ACN

RT (min): 2.05; M+H: 503.2, Purity: 96.6%

### Step 3: Compound 3

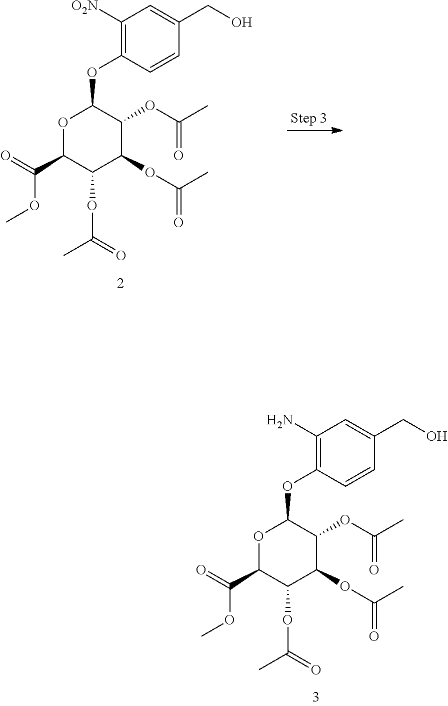 [SMILES: COC(=O)[C@H]1O[C@@H](Oc2ccc(CO)cc2[N+](=O)[O-])[C@H](OC(C)=O)[C@@H](OC(C)=O)[C@@H]1OC(C)=O ; COC(=O)[C@H]1O[C@@H](Oc2ccc(CO)cc2N)[C@H](OC(C)=O)[C@@H](OC(C)=O)[C@@H]1OC(C)=O]

To a stirred solution of compound 2 (8.70 g; 17.21 mmol; 1.00 eq.) in ethyl acetate (100.00 ml; 11.49 V) and THF (100.00 ml; 11.49 V) was added Palladium on carbon (10% w/w) (2.50 g; 2.35 mmol; 0.14 eq.). The reaction mixture stirred for 3 h at RT under hydrogen atmosphere. After completion, the reaction mixture was filtered off through celite. The solvent was concentrated under vacuum to get compound 3 as off-white solid.

Yield: 8.5 g

Percentage Yield: 100%

### Analytical Data:

LCMS: Column: ATLANTIS dC18 (50×4.6 mm) 5 μm; Mobile phase A: 0.1% HCOOH in H2O:ACN (95:5); B: ACN

RT (min): 1.73; M+H: 456.10, Purity: 95.1%

### Step 4: Compound 4

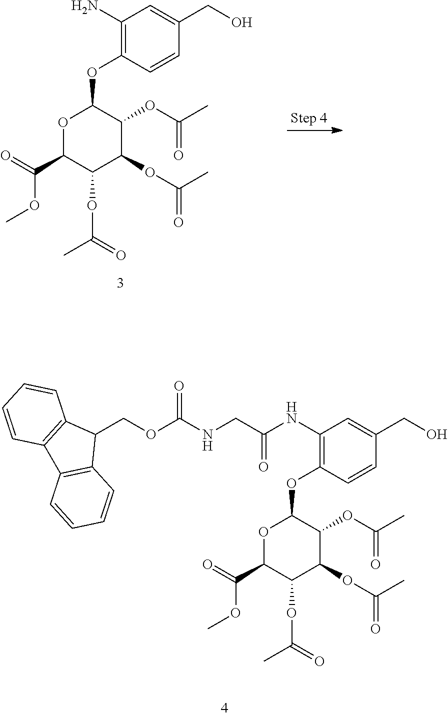 [SMILES: COC(=O)[C@H]1O[C@@H](Oc2ccc(CO)cc2N)[C@H](OC(C)=O)[C@@H](OC(C)=O)[C@@H]1OC(C)=O ; COC(=O)[C@H]1O[C@@H](Oc2ccc(CO)cc2NC(=O)CNC(=O)OCC2c3ccccc3-c3ccccc32)[C@H](OC(C)=O)[C@@H](OC(C)=O)[C@@H]1OC(C)=O]

To a stirred solution of compound 3 (7.60 g; 25.06 mmol; 1.20 eq.) in DCM (250.00 ml; 25.00 V) was added 2-Ethoxy-2H-quinoline-1-carboxylic acid ethyl ester (15.65 g; 62.66 mmol; 3.00 eq.) at 0° C. The reaction mixture was stirred for 16 h at RT. After completion, solvent was removed under reduced pressure to get a crude product. The crude product was purified by column chromatography (56% EtOAc: petroleum ether) to get compound with purity 80%. The compound was purified further by washings with 30% EtOAc and pet ether to get compound 4 as white solid.

Yield: 8.5 g

Percentage Yield: 50.7%

### Analytical Data:

LCMS: Column: ATLANTIS dC18 (50×4.6 mm) 5 μm; Mobile phase A: 0.1% HCOOH in H2O:ACN (95:5); B: ACN

RT (min): 3.03; M+H: 735.2, Purity: 81.9%

### Step 5: Compound 5

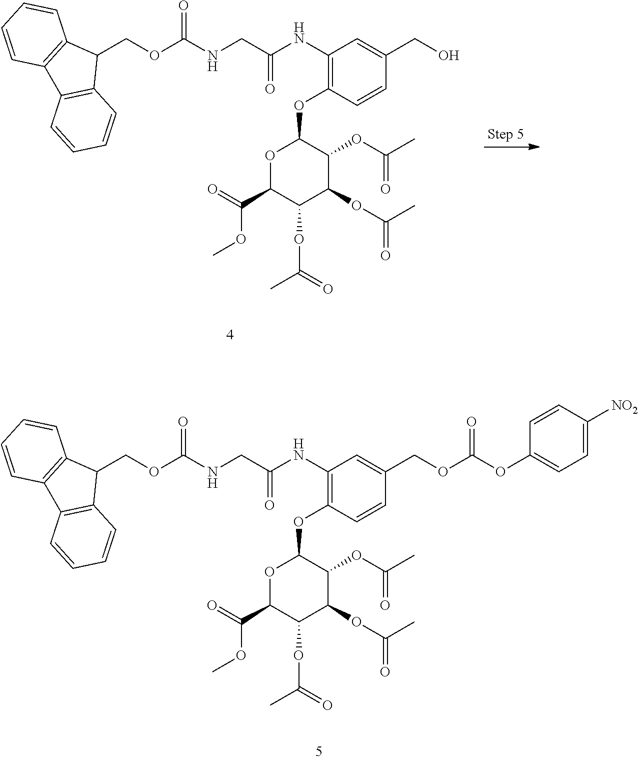 [SMILES: COC(=O)[C@H]1O[C@@H](Oc2ccc(CO)cc2NC(=O)CNC(=O)OCC2c3ccccc3-c3ccccc32)[C@H](OC(C)=O)[C@@H](OC(C)=O)[C@@H]1OC(C)=O ; COC(=O)[C@H]1O[C@@H](Oc2ccc(COC(=O)Oc3ccc([N+](=O)[O-])cc3)cc2NC(=O)CNC(=O)OCC2c3ccccc3-c3ccccc32)[C@H](OC(C)=O)[C@@H](OC(C)=O)[C@@H]1OC(C)=O]

To a stirred solution of compound 4 (2.00 g; 2.49 mmol; 1.00 eq.) in THF (40.00 ml; 20.00 V) at 0° C., were added Carbonic acid bis-(4-nitro-phenyl) ester (3.06 g; 9.97 mmol; 4.00 eq.) and DIPEA (4.40 ml; 24.92 mmol; 10.00 eq.). The reaction mixture was stirred at RT for 12 h. After completion of the reaction, reaction mixture was concentrated under vacuum. The crude product was purified by column chromatography using silica gel (230-400) and pet ether/ethyl acetate as an eluent to afford compound 5 as pale yellow solid.

Yield: 2.0 g

Percentage Yield: 84.6%

### Analytical Data:

LCMS: Column: X-Bridge C8 (50×4.6) mm, 3.5 μm; Mobile phase: A: 0.1% TFA in MilliQ water; B: ACN

RT (min): 3.24; M+H: 900.20, Purity: 94.9%

### Step 6: Compound 6

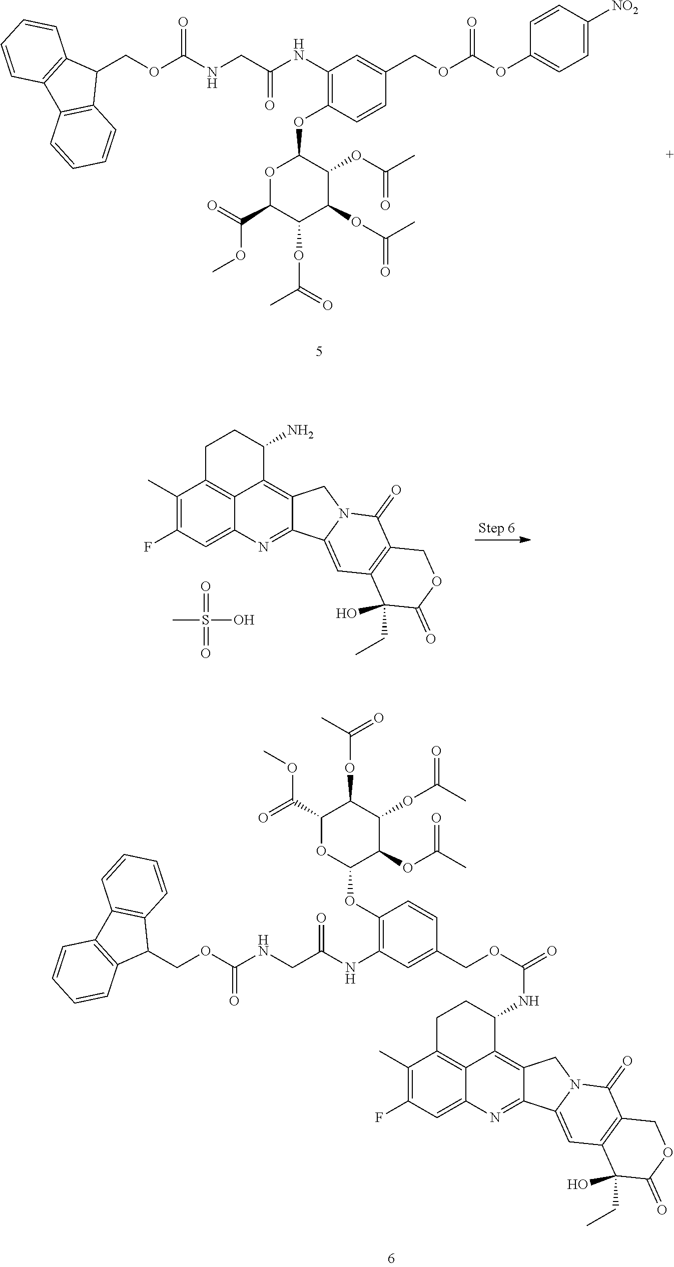

Compound 5 (1,369 g; 1.00 eq.) was dissolved in N,N-dimethylformamide (15.00 ml), Exatecan mesylate (679.7 mg; 1.00 eq.), 4-methylmorpholine for synthesis (0,422 ml; 3.00 eq.) and 1-Hydroxybenzotriazol (172.8 mg; 1.00 eq.) were added. The reaction mixture was stirred at room temperature for overnight. After the stirring time the reaction suspension was changed to a brown solution. The reaction was monitored by LC-MS, which showed a complete conversion of the starting material. The reaction mixture was purified via RP flash chromatography. The product containing fractions were combined, concentrated in vacuo and lyophilized overnight to afford compound 6 as an yellow solid.

Yield: 1.59 g

Percentage Yield: 87.5%

### Analytical Data:

LCMS: Column: Chromolith HR RP-18e (50-4.6 mm); Mobile phase A: 0.05% HCOOH in H2O; B: 0.04% HCOOH and 1% H₂O in ACN; T: 40° C.; Flow: 3.3 ml/min; MS: 100-2000, amu positive; 1%->100% B: 0->2.0 min; 100% B: 2.0->2.5 min

RT (min): 1.95; M+H: 1196.40, Purity: 84.4%.

### Step 7: Compound 7.

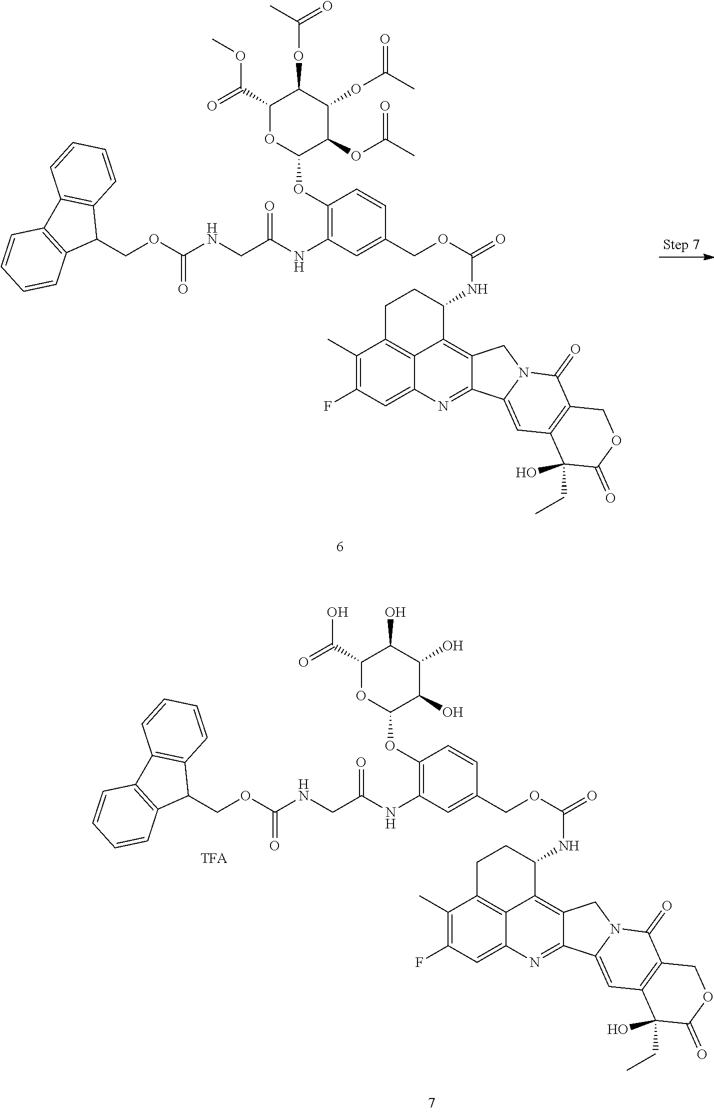 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)OC)[C@@H](OC(C)=O)[C@H](OC(C)=O)[C@H]2OC(C)=O)c(NC(=O)CNC(=O)OCC2c4ccccc4-c4ccccc42)c1)CC3 ; CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CNC(=O)OCC2c4ccccc4-c4ccccc42)c1)CC3]

Compound 6 (1,586 g; 1.00 eq.) was dissolved in tetrahydrofuran (50.00 ml) and a solution (0.1M) of LiOH (contains Lithium hydroxide hydrate (281.77 mg; 6.00 eq.) in water (67,100 ml)) was added dropwise at 0° C. The pH value was checked during the addition. The pH should not exceed 10. The addition of the solution of LiOH was completed after 1.5 hours. The reaction was monitored by LC-MS, which showed a complete conversion of the starting material. The reaction was quenched with citric acid solution, pH adjusted to 5. The reaction mixture was concentrated under reduced pressure. The crude was purified by prep. HPLC. The product containing fractions were combined and lyophilized to afford compound 7 as a dark yellow solid.

Yield: 728 mg

Percentage Yield: 54.8%

### Analytical Data:

LCMS: Column: Chromolith HR RP-18e (50-4.6 mm); Mobile phase A: 0.05% HCOOH in H2O; B: 0.04% HCOOH and 1% H₂O in ACN; T: 40° C.; Flow: 3.3 ml/min; MS: 100-2000, amu positive; 1%->100% B: 0->2.0 min; 100% B: 2.0->2.5 min

RT (min): 1.68; M+H: 1056.30, Purity: 98.5%.

### Step 8: Compound 8

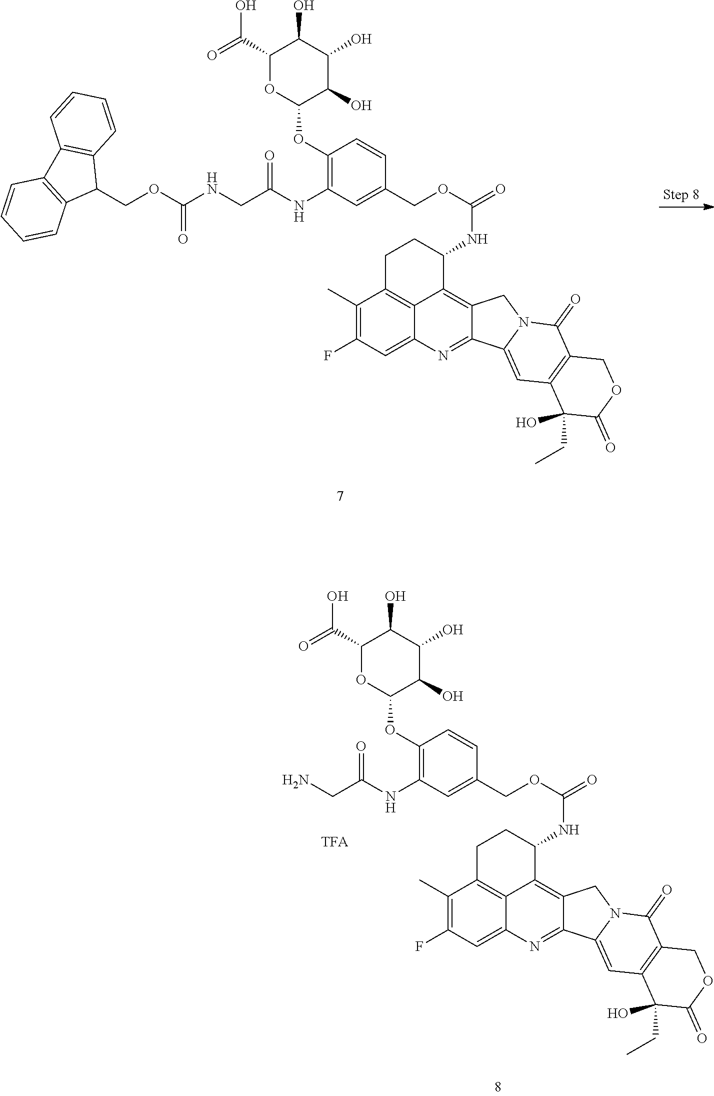 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CNC(=O)OCC2c4ccccc4-c4ccccc42)c1)CC3 ; CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CN)c1)CC3]

Compound 7 (728,000 mg; 1.00 eq.) was dissolved in N,N-dimethylformamide (20.00 ml). Piperidine (136,513 μl; 2.00 eq.) was added and the solution was stirred at RT for totally 4 hours. The reaction was monitored by LC-MS, which showed a complete conversion of the starting material. The reaction mixture was concentrated under reduced pressure and the crude product was purified by RP flash chromatography. The product containing fractions were combined, the solvent was removed partially and it was lyophilized overnight to afford compound 8 as an yellow solid.

Yield: 706 mg

Percentage Yield: 100%

### Analytical Data:

LCMS: Column: Chromolith HR RP-18e (50-4.6 mm); Mobile phase A: 0.05% HCOOH in H2O; B: 0.04% HCOOH and 1% H₂O in ACN; T: 40° C.; Flow: 3.3 ml/min; MS: 100-2000, amu positive; 1%->100% B: 0->2.0 min; 100% B: 2.0->2.5 min

RT (min): 1.22; M+H: 834.30, Purity: 97.6%.

### Step 9: Compound 9

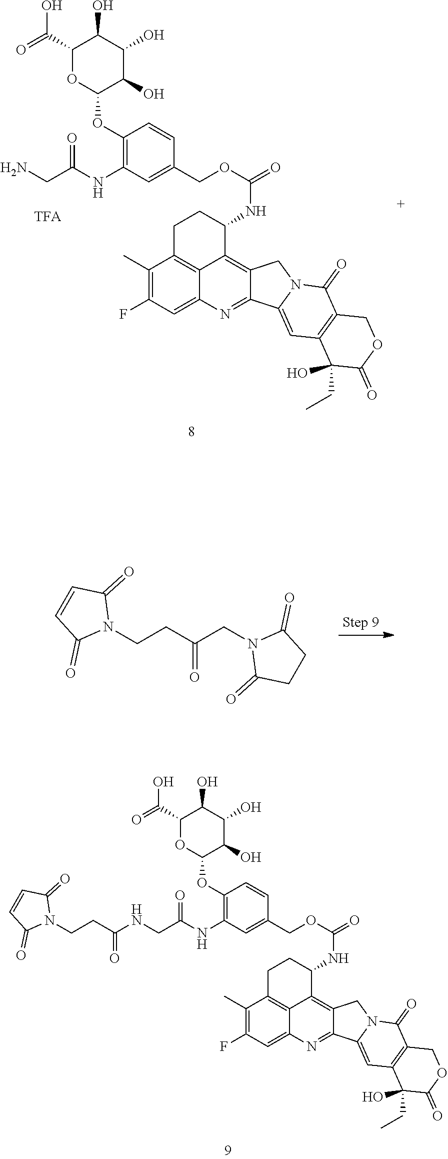 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CN)c1)CC3 ; O=C(CCN1C(=O)C=CC1=O)CN1C(=O)CCC1=O ; CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CNC(=O)CCN2C(=O)C=CC2=O)c1)CC3]

To a solution of compound 8 (854 mg; 1.00 eq.) in dimethylformamid (30.00 ml) were added N-ethyldiisopropylamine (149,234 μl; 1.00 eq.) and 3-(2,5-Dioxo-2,5-dihydro-pyrrol-1-yl)-propionic acid 2,5-dioxo-pyrrolidin-1-yl ester (233.61 mg; 1.00 eq.). The reaction mixture was stirred at RT for 3 hours. The reaction was monitored by LC-MS, which showed a complete conversion of the starting material. The reaction mixture was concentrated under reduced pressure and the crude product was by RP flash chromatography. The product containing fractions were combined, concentrated and lyophilized to give the desired produce with a purity of 91%. This material was again purified by RP chromatography to give compound 9 as an yellow solid.

Yield: 580 mg

Percentage Yield: 60.1%

### Analytical Data:

LCMS: Column: Chromolith HR RP-18e (50-4.6 mm); Mobile phase A: 0.05% HCOOH in H2O; B: 0.04% HCOOH and 1% H₂O in ACN; T: 40° C.; Flow: 3.3 ml/min; MS: 100-2000, amu positive; 1%->100% B: 0->2.0 min; 100% B: 2.0->2.5 min

RT (min): 1.38; M+H: 985.30, Purity: 90% (the other 10% of isomer can be removed by HPLC)

¹H NMR (500 MHZ, DMSO-d6) δ 13.10-12.44 (m, 1H), 9.08 (s, 1H), 8.32 (t, J=5.8 Hz, 1H), 8.16 (s, 1H), 8.02 (d, J=8.8 Hz, 1H), 7.76 (d, J=10.9 Hz, 1H), 7.31 (s, 1H), 7.15-7.09 (m, 2H), 6.98 (s, 2H), 5.48-5.38 (m, 2H), 5.32-5.22 (m, 3H), 5.11-5.01 (m, 2H), 4.87 (d, J=7.6 Hz, 1H), 3.92-3.88 (m, 1H), 3.89-3.84 (m, 2H), 3.65-3.61 (m, 2H), 3.46-3.41 (m, 1H), 3.42-3.37 (m, 1H), 3.38-3.31 (m, 1H), 3.28-3.20 (m, 1H), 3.15-3.07 (m, 1H), 2.48-2.44 (m, 2H), 2.38 (s, 3H), 2.24-2.13 (m, 2H), 1.94-1.80 (m, 2H), 0.88 (t, J=7.3 Hz, 3H).

### Example 1B: Synthesis of a Drug-Linker Compound with Legumain-Cleavable Linker: Drug-Linker Compound 2 (DL2)

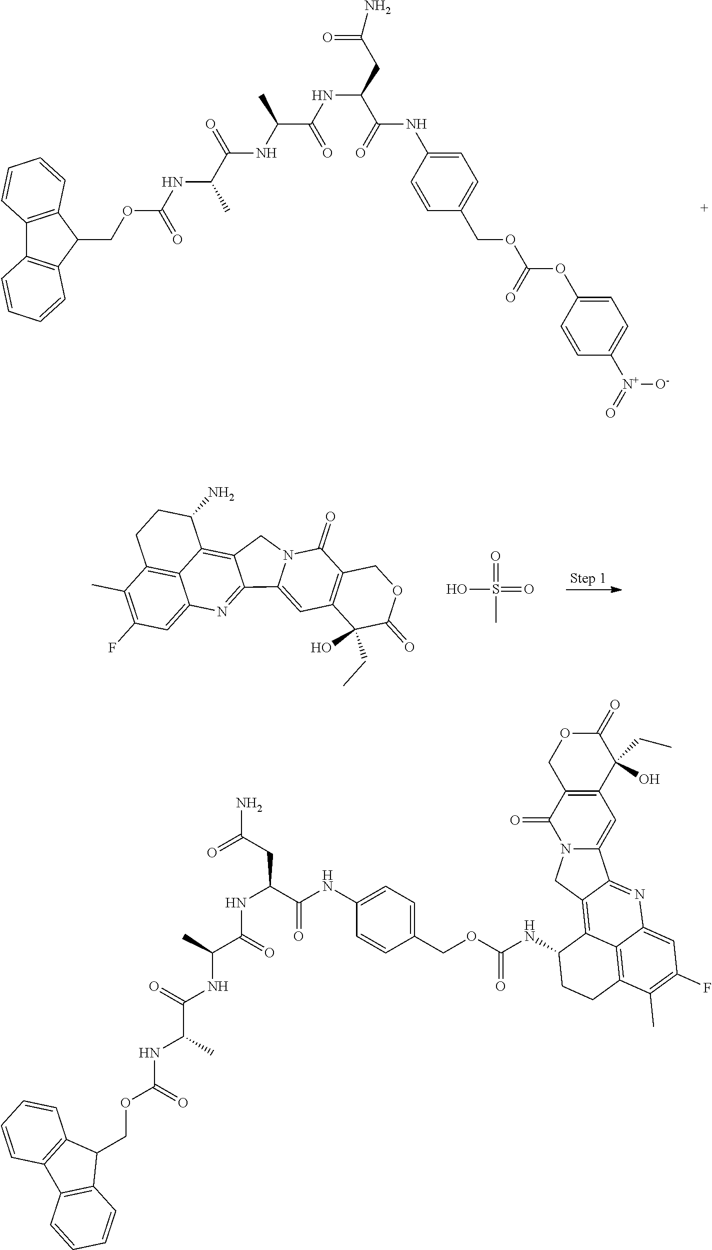

### Step 1

{-3-carbamoyl-2-[(2S)-2-[(2S)-2-({[(9H-fluoren-9-yl) methoxy]carbonyl}amino)propanamido]propanamido]propanamido]phenyl}methyl 4-nitrophenyl carbonate (400 mg; 0.52 mmol; 1.00 eq.) [commercially available from Levena Biopharma US] was dissolved in N,N-Dimethylformamide (5.00 ml). (10S,23S)-23-amino-10-ethyl-18-fluoro-10-hydroxy-19-methyl-8-oxa-4,15-diazahexacyclo[14.7.1.0²,¹4.04,¹³.06,¹¹.0²⁰,²4]tetracosa-1,6(11),12,14,16,18,20(24)-heptaene-5,9-dione; methanesulfonic acid (277.30 mg; 0.52 mmol; 1.00 eq.), N-Ethyldiisopropylamine (0.27 ml; 1.57 mmol; 3.00 eq.) and 1-Hydroxybenzotriazol (HOBT) (3.52 mg; 0.03 mmol; 0.05 eq.) were added. The reaction mixture was stirred at room temperature overnight. LC/MS indicated complete conversion.

The crude reaction mixture was purified via prep HPLC and lyophilized yielding 365 mg (0.343 mmol) of {4-[(2S)-3-carbamoyl-2-[(2S)-2-[(2S)-2-({[(9H-fluoren-9-yl) methoxy]carbonyl}amino) propanamido]propanamido]propanamido]phenyl}methyl N-[(10S,23S)-10-ethyl-18-fluoro-10-hydroxy-19-methyl-5,9-dioxo-8-oxa-4,15-diazahexacyclo[14.7.1.0²,¹4.04,¹³.06,¹¹.0²⁰,²4]tetracosa-1,6(11),12,14,16,18,20(24)-heptaen-23-yl]carbamate.

LC/MS: [M+H]=1064.2

Prep HPLC:

Column: sunfire prep c18 obd-75.0 g (250 bar)

Solvent A: Wasser 0.1% TFA Solvent C:

Solvent B: Acetonitril 0.1% TFA

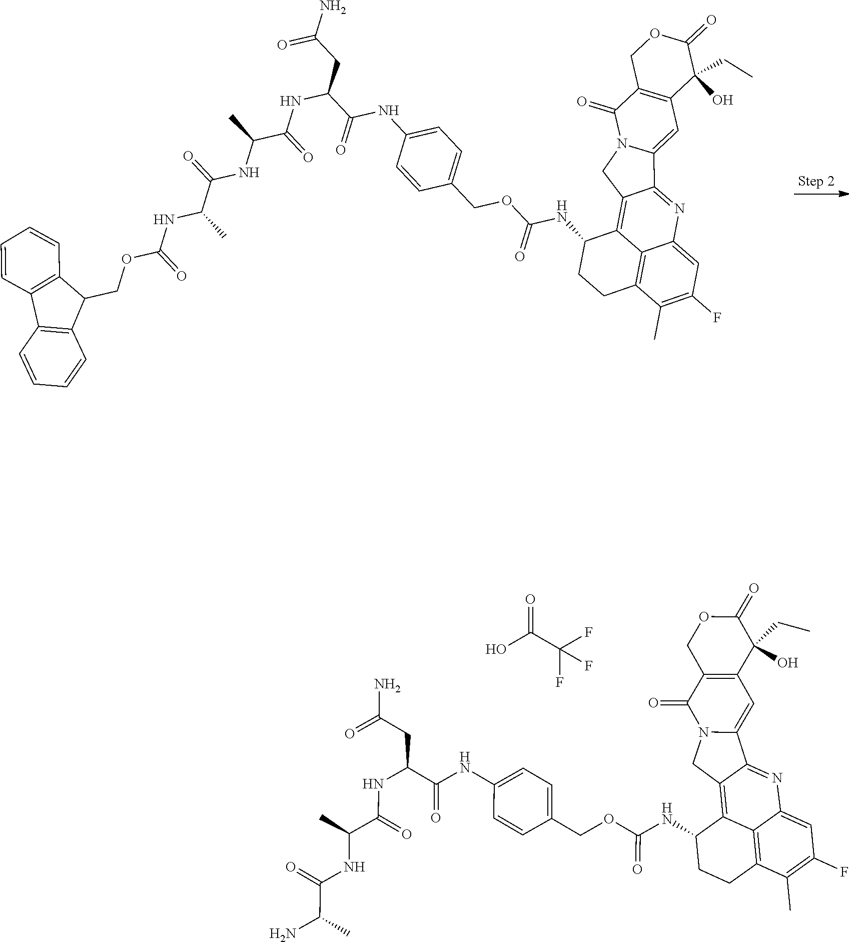 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CC(N)=O)NC(=O)[C@H](C)NC(=O)[C@H](C)NC(=O)OCC2c4ccccc4-c4ccccc42)cc1)CC3 ; CC[C@@]1(O)C(=O)OCc2c1cc1n(c2=O)Cc2c-1nc1cc(F)c(C)c3c1c2[C@@H](NC(=O)OCc1ccc(NC(=O)[C@H](CC(N)=O)NC(=O)[C@H](C)NC(=O)[C@H](C)N)cc1)CC3 ; O=C(O)C(F)(F)F]

### Step 2

{4-[(2S)-3-carbamoyl-2-[(2S)-2-[(2S)-2-({[(9H-fluoren-9-yl) methoxy]carbonyl}amino) propanamido]propanamido]propanamido]phenyl}methyl N-[(10S,23S)-10-ethyl-18-fluoro-10-hydroxy-19-methyl-5,9-dioxo-8-oxa-4,15-diazahexacyclo[14.7.1.0²,¹4.04,¹³.06,¹¹.0²⁰,²4]tetracosa-1,6(11),12,14,16,18,20(24)-heptaen-23-yl]carbamate (365 mg; 0.34 mmol; 1.00 eq.) was dissolved in N,N-(4.00 ml). Piperidine for synthesis (0.07 ml; 0.69 mmol; 2.00 eq.) was added and the reaction solution was stirred at rt for 1 h.

The reaction mixture was purified via prep HPLC yielding 300 mg (0.314 mmol) of trifluoroacetic acid; {4-[(2S)-2-[(2S)-2-[(2S)-2-aminopropanamido]propanamido]-3-carbamoylpropanamido]phenyl}methyl N-[(10S,23S)-10-ethyl-18-fluoro-10-hydroxy-19-methyl-5,9-dioxo-8-oxa-4,15-diazahexacyclo[14.7.1.0²,¹4.04,¹³.06,¹¹.0²⁰,²4]tetracosa-1,6(11),12,14,16,18,20(24)-heptaen-23-yl]carbamate.

LC/MS: [M+H]: 841.3

Prep HPLC for purification:

RediSep Säule: C18 130 g

SN: E0410A0D24BE1 Lot: 262118923W

Flowrate: 75 ml/min

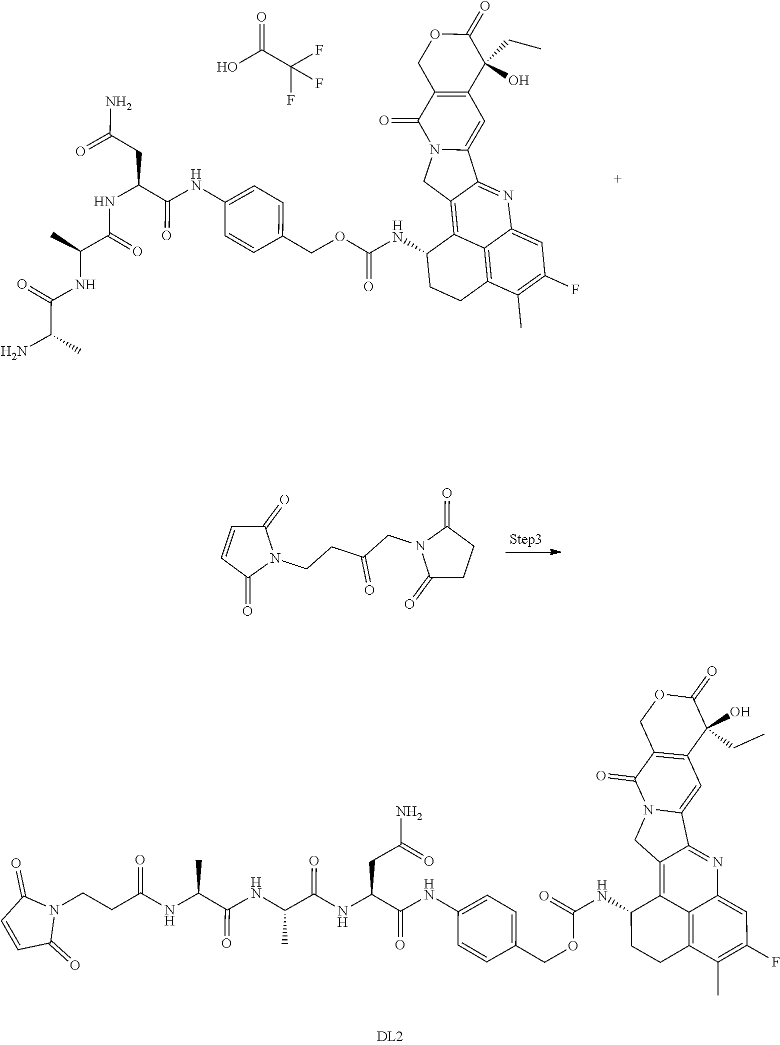 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CC(N)=O)NC(=O)[C@H](C)NC(=O)[C@H](C)N)cc1)CC3 ; O=C(O)C(F)(F)F ; O=C(CCN1C(=O)C=CC1=O)CN1C(=O)CCC1=O ; CC[C@@]1(O)C(=O)OCc2c1cc1n(c2=O)Cc2c-1nc1cc(F)c(C)c3c1c2[C@@H](NC(=O)OCc1ccc(NC(=O)[C@H](CC(N)=O)NC(=O)[C@H](C)NC(=O)[C@H](C)NC(=O)CCN2C(=O)C=CC2=O)cc1)CC3]

### Step 3

To a solution of trifluoroacetic acid {4-[(2S)-2-[(2S)-2-[(2S)-2-aminopropanamido]propanamido]-3-carbamoylpropanamido]phenyl}methyl N-[(10S,23S)-10-ethyl-18-fluoro-10-hydroxy-19-methyl-5,9-dioxo-8-oxa-4,15-diazahexacyclo[14.7.1.0²,¹4.04,¹³.06,¹¹.0²⁰,²4]tetracosa-1,6(11),12,14,16,18,20(24)-heptaen-23-yl]carbamate (571 mg; 0.60 mmol; 1.00 äq.) in N,N-Dimethylformamide (20 ml) was added NEthyldiisopropylamine (203 μl; 1.20 mmol; 2.00 eq.) and N-Succinimidyl 3-maleimidopropionate (162 mg; 0.60 mmol; 1.00 eq.). The reaction mixture was then stirred for 10 min and monitored via LC/MS.

The reaction mixture was purified via prep HPLC yielding 378 mg (0.35 mmol) of DL2.

LC/MS: [M+H]: 992.4

Prep HPLC for purification:

RediSep column: C18 86 g

SN: E0410A8B46130 Lot: 281729189W

Flowrate: 60 ml/min

Condition-Volumen: 264.0 ml

Eluent 1: A1 WATER 0.1% TFA

Eluent 2: B1 ACETONITRILE 0.1% TFA

Analytics for sequence:

Method Info: A: H₂O+0.05% HCOOH|B: MeCN+0.04% HCOOH+1% H2O

T: 40° C.|Flow: 3.3 ml/min|MS: 100-2000 amu positive

Column: Chromolith HR RP-18e 50-4.6 mm

0%->100% B: 0->2.0 min|100% B: 2.0->2.5 mi

### Example 2: Preparation of an Immunoconjugate: A Glucuronide-Based Conjugate of mAb1 (Referred to as ADC1)

### Antibody Preparation and Conjugation

The monoclonal antibody (mAb) was thawed at 2-8° C. up to 3 days prior to conjugation and stored at 2-8° C. until use. The mAb was equilibrated at room temperature on the day of conjugation prior to use. The mAb solution (25.3 mg/mL) in 25 mM Sodium acetate pH 5.5 was pH adjusted by addition of 4% v/v of 0.5 M Tris, 0.025 EDTA, pH 8.5. Subsequently, interchain disulfides were reduced incubating the pH adjusted mAb solution with 5 molar equivalents (relative to the mAb) of TCEP at 20° C. for 2 h. Afterwards, the solution was incubated with 10 molar equivalents (relative to the mAb) of drug-linker (DL) for 1 h at 20° C. to conjugate the DL to the antibody. The reaction was stopped by addition of 10 molar equivalents (relative to the mAb) of 20 mM N-acetyl cysteine (NAC) for 20 min at 20° C. The pH was again adjusted by addition of 5% v/v of 0.15 M acetic acid and vacuum filtered using a 0.22 μm PES filter unit. The reaction mixture was diafiltrated via TFF using Pellicon® Capsule with Ultracel® Membrane, C Screen, 0.1 m². As diafiltration buffer 10 mM Histidine, 40 mM NaCl at pH 5.5 was used and 15 diafiltration volumes (DVs) with a flowrate of 5 L/min/m² were needed. After TFF the sample was again filtered using a 0.22 μm PES filter unit and diluted to a concentration of 15 mg/ml. Final formulation of the ADC was 10 mM Histidine, 40 mM NaCl, 6% Trehalose, pH 5.5 at 10 mg/ml.

### Quality Attributes:

| Endotoxin | <0.01 EU/mg |
|---|---|
| % Monomer | 95.4 |
| DAR (PLRP) | 7.8 |
| % mol/mol residual Toxin | 0.5 |
| [P] by UV-vis | 10.1 |
| Volume (ml) | 339.8 |
| Amount (mg) | 3432 |

### Drug Substance Characterization

### Size Exclusion Chromatography (SEC) Method

Monomer content and purity were assessed by size exclusion chromatography on a TOSOH TSKgel G3000SWXL 7.8 mm×30 cm, 5 μm with a security guard column run at 0.5 ml/min in 10% IPA, 0.2M Potassium phosphate, 0.25M Potassium chloride, pH 6.95 running isocratically for 30 mins. Injection volumes ranged from 1-10 μl (up to 20 μg protein).

SEC Method Parameters

| Wavelength | 280 | nm |
|---|---|---|
| Column | TOSOH TSKgel G3000SWXL | |
| | 7.8 mm × 30 cm, 5 μm | |
| Mobile Phase | 10% IPA, 0.2M Potassium phosphate, | |
| | 0.25M Potassium | |
| 1. | chloride, pH 6.95 | |
| Injection Volume | 1-20 μL (5-20 μg of protein) | |
| Column Temperature | 25° | C. |
| Gradient | Isocratic elution | |
| Flow rate | 0.5 | mL/min |
| Run Time | 30 | min |

Typical SEC chromatogram showing the purity of the stock mAb and the final BDS:
For the BDS material shown above, 4.6% HMWS and a monomeric purity of 95.4% have been reported.

### Reversed-Phase HPLC (RP HPLC) Method

Reverse phase was performed on a Polymer Labs PLRP-S 2.1 mm×50 mm, 5 μm, 1000 Å column run at 1.0 mL/min/80° C. with a 25-minute linear gradient between 0.1% TFA 25% CH3CN (mobile phase B) and 0.1% TFA 50% CH3CN (A). Both reduced antibody and fully conjugated ADCs were analyzed by non-reducing PLRP conditions. The method used for sample preparation is given in the following table:

| Time (min) | % B* | FR (ml/min) | HPLC Setup |
|---|---|---|---|
| 0 | 22.5 | 1 | Column Temperature: 80° C. |
| 2 | 22.5 | 1 | Detection Wavelength: 280 nm, |
| | | | 214 nm, 252 nm, 320 nm: 16 nm |
| | | | bandwidth |
| 21.5 | 49.5 | 1 | Reference: 440 nm, 80 nm |
| 22.5 | 90.0 | 1 | Peak width: >0.4 min, slit 8 nm |
| 26.5 | 90.0 | 1 | Report: 214 nm |
| 27.5 | 22.5 | 1 | Collect spectrum between 200 and |
| | | | 600 nm, Step 1.2 nm |
| 32.0 | 22.5 | 1 | Non-reducing PLRP sample |
| | | | preparation. |
| | | | 10 μg mAb/ADC diluted in 0.5M |
| | | | Tris/HCl pH 8.0 (volume 50 μl). |
| | | | Mix with 50 μl of 49% |
| | | | ACN: 49 % H₂O: 2 % FA. |
| | | | Inject 20 μl. Report data at 214 nm. |

Typical RP-HPLC chromatogram showing the separation of light and heavy chains. The chromatogram below shows an overlay of the mAb and the final BDS:
For the BDS material above, a DAR of 7.8 was reported.

### Hydrophobic Interaction Chromatography

Hydrophobic Interaction Chromatography (HIC) with gradient elution and detection by absorbance at 214 nm was used to assess the average DAR of the ADC. The ADC molecules are described in order of increasing DAR by decreasing the salt concentration in the mobile phase consisting of 1.5 M Ammonium sulphate/20 mM Sodium phosphate/pH 6.95. As column a Tosoh TSKgel Butyl-NPR Column, 4.6×35 mm, 2.5 μm was used. Injection volumes ranged from 1-10 μl for antibody/conjugate samples (5-20 μg protein).

| Parameter | Values | | |
|---|---|---|---|
| Mobile Phase A | 1.4M (NH₄)₂SO₄ / 25 mM NaPᵢ/ pH 6.95 ± 0.05 | | |
| Mobile Phase B | 25 mM NaPᵢ / pH 6.95 ± 0.05 + 25% IPA | | |
| | Time | % B | Flow Rate |
| Gradient | 0 | 0 | 0.8 ml/min |
| | 12 | 100 | 0.8 ml/min |
| | 12.1 | 0 | 0.8 ml/min |
| | 18 | 0 | 0.8 ml/min |

| Column temperature | 25° C. | |
|---|---|---|
| Autosampler | 4° C. | |
| | Wavelength | Slit Width |
| Detection | 280 nm | 16 nm |
| | 252 nm | 16 nm |
| | 214 nm | 16 nm |

| | |
|---|---|
| | Reference: 440 nm (80 nm slit width) |
| Spectra | 220 to 600 nm with 1.2 step size |

Typical HIC chromatogram showing the separation of mAb and ADC. The chromatogram below shows an overlay of the input mAb and the final BDS:

### Free-Drug Method

Reverse Phase HPLC was performed on a Phenomenex Kinetex Core Shell 2.6 μm C8 Column, 100 Å, 50×4.6 mm column run at 2 mL/min, 60° C. with an 8 min linear gradient between 95% A (0.05% TFA/H2O) to 95% B (0.05% TFA/CH3CN). Toxin linker standards were run as NAC quenched. Data were collected at 214 nm and spectral analysis at 252, 360 and 280 nm to monitor the linker chromophore and any protein residue in the sample. Samples were deproteinated by cold methanol/salt extraction prior to HPLC analysis: 2 μl 5 M NaCl was added to 50 μl samples, followed by 150 μl ice-cold methanol. Samples were vortexed and incubated at −20° C. for 30 mins. Precipitated protein was pelleted by centrifugation at 15000×g, 4° C., 30 mins. 125 μl supernatant was diluted with 125 μl Elga 18.2 MQ water and mixed. 100 μl sample was injected and all data reported at 360 nm. The level of residual toxin related species was calculated relative to a standard curve of toxin. The amount of toxin observed was reported as a % mole/mole value, relative to the total toxin content of the sample.

RP-HPLC chromatogram overlay (360 nm) of crude conjugate reaction (blue), DVO (red), DV6 (green),
DV10 (pink) and BDS PPB-13430 (olive green).

### Endotoxin

Endotoxin was determined by kinetic chromogenic LAL assay using an Endosafe PTS endotoxin system (Charles River). Buffers and antibodies were diluted 10-fold in LAL reagent water. The ADCs were diluted 10-fold in LAL reagent water. All samples were analyzed on 0.01-1 EU/mL cartridges. The EU/mL value was converted to EU/mg by dividing by the ADC [P] mg/mL.

### GD2-ADCs: In Vitro and In Vivo Experimental Results

### Experiment 1. Dose Response Efficacy Study of Anti-GD2 ADC in a Neuroblastoma (CHP134) Xenograft Model

The dose-response relationship of the anti-GD2 ADC Molecule 1 was evaluated in CHP134, a human neuroblastoma xenograft tumor model which expresses high levels of GD2.

Female athymic nude mice (nu/nu) with established CHP134 tumors (˜150 mm3) were treated with an intravenous (IV) injection of Molecule 1 at doses ranging from 0.25 mg/kg to 1.0 mg/kg at weekly schedule (qw) for 4 weeks.

In this study, treatment was well tolerated with no toxicity and normal weight gain observed (FIG. 1). The effects of treatment on CHP134 tumor growth and the individual tumor sizes on the day the vehicle control treated tumors reached the study endpoint (>1,000 mm3) are illustrated in FIGS. 2. Molecule 1 administered at 0.25, 0.5, and 1.0 mg/kg at DO (Day0) D7, D14, and D21 demonstrated dose dependent anti-tumor activity. The lowest dose tested, 0.25 showed efficacy of 59% tumor growth inhibition (TGI) which was still significant (p<0.01), (FIG. 2). Strong activity inducing tumor regression was observed with 0.5 mg/kg and 1.0 mg/kg Molecule 1, which was highly significant (p<0.0001, Day18) for both doses (FIG. 2). At the end of the study (Day85), complete responses were observed in all animals treated with doses of 0.5 mg/kg and 1.0 mg/kg (16 out of 16) of Molecule 1 (FIG. 3).

In conclusion, results from this study demonstrated potent anti-tumor activity of Molecule 1 resulting in significant efficacy and tumor regression in CHP134 tumors. The minimum efficacious dose (MED), defined as the lowest dose to induce a >20% decrease in tumor volume from baseline (for any time point post treatment initiation), was determined to be approximately 0.5 mg/kg of Molecule 1 in the CHP134 tumor model.

### Experiment 2. Efficacy Study of Anti-GD2 ADC in an Osteosarcoma PDX Model

The efficacy of the anti-GD2 ADC Molecule 1 was evaluated in the CTG-2735 osteosarcoma PDX model which expresses GD2.

Female athymic nude mice (nu/nu) with established CTG-2735 tumors (˜250 mm3) were treated with a single IV injection of Molecule 1 at dose of 10.0 mg/kg at Day0. The group size for the vehicle group and the treatment group was 3.

In this study, treatment was well tolerated with no toxicity and normal weight gain observed (FIG. 4). Treatment with 10 mg/kg Molecule 1 caused strong tumor growth inhibition resulting in long lasting tumor regressions until day 62, when the experiment was ended (See, FIGS. 5 and 6).

In conclusion, results from this study demonstrated potent anti-tumor activity of Molecule 1 in the osteosarcoma PDX model CTG-2735.

### Experiment 3. Efficacy Study of Anti-GD2 ADC in a SCLC PDX Model

The efficacy of the anti-GD2 ADC Molecule 1 was evaluated in the CTG-0199 SCLC PDX model which expresses GD2.

Female athymic nude mice (nu/nu) with established CTG-0199 tumors (˜250 mm3) were treated with a single IV injection of Molecule 1 at dose of 10.0 mg/kg at Day0. The group size for the vehicle group and the treatment group was 5.

In this study, treatment was well tolerated with no toxicity and normal weight gain observed (FIG. 7). Treatment with 10 mg/kg Molecule 1 caused strong tumor growth inhibition resulting in long lasting tumor regressions until day 53 when the experiment was ended (FIG. 8, 9).

In conclusion, results from this study demonstrated potent anti-tumor activity of Molecule 1 in the SCLC PDX model CTG-0199.

### Experiment 4. Combination Efficacy Study of Anti-GD2 ADC and ATR Inhibitor in an Osteosarcoma (CTG-2264) Patient-Derived Xenograft (PDX) Model

The antitumor activities of selected ADCs of the present invention were tested in the CTG-2264, osteosarcoma PDX model.

Female athymic nude mice (nu/nu) with established CTG-2264 tumors (˜250 mm3) were treated either with a single IV injection of Molecule 1 at dose of 3.0 mg/kg at Day0, with the ATR inhibitor at dose of 10 mg/kg, orally, (p.o.), daily (qd); and with the combination of both treatments. Vehicle control was saline and dosed (10 mL/kg) at Day0. The group size for each treatment group was 3. The calculation of the Tumor Growth Inhibition (TGI) was done for Day 23 after treatment start, when the mean tumor volume of the control group reached 1000 mm3 (see FIG. 11, 12).

Treatments were well tolerated, with no toxicity or clinical signs noted and no mean body weight loss compared to the initial weight was observed for all treatment groups (FIG. 10). These ADCs, alone and in combination, caused tumor growth inhibition (TGI) of 65%, 67% and −72% (regression) in CTG-2264 osteosarcoma PDX model.

In conclusion, the above referenced combination showed benefits compared to either of the two mono-therapeutics, 10 mg/kg (p<0.0001).

### Experiment 5. ADC Cell Killing Assay on Human Cancer Cell Lines

The in vitro potency of the ADCs hu14.18-IgG1.4 (K322A)-delK-βGluc Exatecan and rituximab IgG1 βGluc Exatecan, the unconjugated antibody hu14.18-IgG1.4 (K322A)-delk and the free payload Exatecan was measured on antigen-positive human tumor cell lines CHP-134 (neuroblastoma, #ACC653, DSMZ, Braunschweig, Germany), NCI-H446 (small cell lung cancer (SCLC), #HTB-171, ATCC, Manassas, VA, USA) as well as M21 (melanoma, Scripps Research Institute, La Jolla, CA, USA) and antigen-negative human tumor cell line MDA-MB-468 (breast carcinoma, #ACC738, DSMZ, Braunschweig, Germany) using the CellTiter Glo® Luminescent Cell Viability assay (#G7573, Promega Corporation, Madison, WI, USA).

The cell lines CHP-134, NCI-H446 and MDA-MB-468 were cultured as instructed by vendor and maintained in RPMI1640 medium with GlutaMAX™ supplement (#61870-010, Gibco™, purchased from Thermo Fisher Scientific, Waltham, MA, USA), 1 mM sodium pyruvate (#11360-070, Gibco™, Thermo Fisher Scientific) and 10% fetal bovine serum (FBS) (#S0615, Sigma Aldrich, St. Louis, MO, USA). The melanoma cell line M21 was cultured in DMEM with stable L-glutamine (#41965-039, Gibco™, Thermo Fisher Scientific) and 10% FBS.

The day before treatment, 625 cells/well (90 μl) (CHP-134, M21 or MDA-MB-468) or 1250 cells/well (90 μl) (NCI-H446) were plated in sterile Falcon™ 96-well, cell culture-treated, flat bottom microplates (#353219, Corning, NY, USA). After overnight incubation at 37° C. and 5% CO₂ or 10% CO₂, a 10-fold starting concentration of the compound and a serial dilution (1:4) was prepared using the cell culture medium or RPMI1640 medium including supplements stated above. A total of 10 μl of compound solution was added per well (technical triplicates). Control wells were treated with a respective amount of dimethylsulfoxid (control wells for free payload) or cell culture medium/RPMI 1640 (control wells for ADCs and unconjugated antibody as well as background wells containing no cells). Following 6 days of incubation, 100 μl Cell Titer-Glo® reagent was added to each well and plates were incubated for 2 min with shaking at 300 rpm and for additional 20 min at room temperature (protected from light). Afterwards, luminescent signal was measured on a Varioskan Flash or Varioskan Lux plate reader (Thermo Fisher Scientific) to determine cell viability. Relative light units (RLU) were processed by subtracting the background and by converting to % viability (whereas RLU of untreated control cells is defined as 100%) or % effect (calculated by subtracting 100% from % viability). The processed data was used to describe the dose-response by % effect vs. concentration [M] with the equation log (inhibitor) vs. response-variable slope (four parameters) (GraphPad Prism (version 8.2.0) for Windows, GraphPad software, La Jolla California USA, www.graphpad.com). Data were displayed with error bars indicating the standard deviation (SD) of technical triplicates. Several experiments were performed and geometric mean values of determined IC50s (geomean IC50 [nM]) was calculated.

The cytotoxic activity of the anti-GD2 hu14.18-IgG1.4 (K322A)-delk-βGluc Exatecan ADC (Molecule 1) was determined on target-positive and target-negative human cancer cell lines. The rituximab IgG1 βGluc Exatecan ADC with the same linker-payload and comparable DAR (Molecule 2) and the unconjugated antibody hu14.18-IgG1.4 (K322A)-delK (Molecule 3) were used as control. To confirm a general sensitivity of human cancer cell lines to the free payload, Exatecan (Molecule 4) was included in the testing as well.

The results of the in vitro activity are summarized in Table 1 and representative dose-response curves are displayed in FIG. 1—Figure. The anti-GD2 hu14.18-IgG1.4 (K322A)-delk-βGluc Exatecan ADC (Molecule 1) showed effective inhibition of cell viability and a specific, sub-nanomolar or single-digit nanomolar potency on GD2+ expressing cancer cell lines CHP-134 (FIG. 1), M21 (Figure) and NCI-H446 (Figure) as shown in relation to the non-binding control ADC (Molecule 2). For the target-negative cell line MDA-MB-468 (Figure), which showed comparably high sensitivity to the free payload Exatecan (Molecule 4), no specific cytotoxic effect was observed for Molecule 1 compared to non-binding control ADC (Molecule 2).

Treatment of the GD2-positive neuroblastoma cell line CHP-134 with the unconjugated antibody hu14.18-IgG1.4 (K322A)-delk (Molecule 3) showed cell killing activity at highest concentrations tested (Figure). This effect is in accordance with data published by Horwacik et al. (Cancer Letters, Volume 341, Issue 2, 2013, pp. 248-264), reporting a negative effect on CHP-134 cell viability after treatment with anti-GD2 mouse mAb 14G2a. A minor effect on cell viability after treatment with hu14.18-IgG1.4 (K322A)-delk (Molecule 3) was observed for SCLC cell line NCI-H446 and melanoma cell line M21 while no effect was determined on target-negative MDA-MB-468 cell line.

| | | CHP- | | NCI- | | M21 | | MDA-MB- | |
|---|---|---|---|---|---|---|---|---|---|
| | | 134GD2- | | H446GD2- | | GD2-positive | | 468GD2- | |
| | | positiveN ≥ 4 | | positiveN = 5 | | N ≥ 6 | | negative N ≥ 3 | |
| Molecule | | IC₅₀ | Span | IC₅₀ | Span | IC₅₀ | Span | IC₅₀ | Span |
| No. | Sample | [nM] | (%)* | [nM] | (%)* | [nM] | (%)* | [nM] | (%)* |
| 1 | Hu14.18- | 0.8 | 100 | 0.7 | 91 | 2.6 | 94 | 28 | 95 |
| | IgG1.4 | | | | | | | | |
| | (K322A)- | | | | | | | | |
| | delK-βGluc | | | | | | | | |
| | Exatecan, | | | | | | | | |
| | DAR 8 | | | | | | | | |
| 2 | Rituximab | 5.4 | 100 | 24 | 90 | >10 | 74 | 13 | 99 |
| | IgG1 βGluc | | | | | | | | |
| | Exatecan, | | | | | | | | |
| | DAR 7.7 or | | | | | | | | |
| | 7.9 | | | | | | | | |
| 3 | Hu14.18- | 110 | 83 | NC | 23 | NC | 36 | No | 4 |
| | IgG1.4 | | | | | | | effect | |
| | (K322A)- | | | | | | | | |
| | delk | | | | | | | | |
| | Exatecan | 0.2 | 100 | 0.07 | 96 | 1.1 | 95 | 0.3 | 100 |

NC: Not calculable due to incomplete dose-response curve
N: number of experiments
IC₅₀: half maximal inhibitory concentration; concentration at which 50% of maximal inhibition was observed; table includes the geomean IC₅₀ [nM] calculated from IC₅₀ values of indicated number of experiments
Span (%): % efficacy at highest test concentration*. Percentage of non-viable, total cells in relation to untreated control cells; table includes the geomean Span (%) calculated from Span (%) values of indicated number of experiments
Highest test concentration: 100 nM for Molecule 1,2,4; 1 μM for Molecule 3

Table 1 Geomean IC50 values [nM] and geomean Span (%) of hu14.18-IgG1.4 (K322A)-delK-βGluc Exatecan, Rituximab IgG1 βGluc Exatecan, hu14.18-IgG1.4 (K322A)-delk and payload Exatecan in human cancer cell lines

### Figure Legends For Experiment 5

FIG. 13 In vitro dose-response curves for hu14.18-IgG1.4 (K322A)-delK-βGluc Exatecan (Molecule 1), control ADC Rituximab IgG1 βGluc Exatecan (Molecule 2) and payload Exatecan (Molecule 4) in CHP-134 cells. One representative dose-response curve is shown as means from triplicate values±SD.

FIG. 14 In vitro dose-response curves for hu14.18-IgG1.4 (K322A)-delK-βGluc Exatecan (Molecule 1), control ADC Rituximab IgG1 βGluc Exatecan (Molecule 2) and payload Exatecan (Molecule 4) treatment in M21 cells. One representative dose-response curve is shown as means from triplicate values±SD.

FIG. 15 In vitro dose-response curves for hu14.18-IgG1.4 (K322A)-delK-βGluc Exatecan (Molecule 1), control ADC Rituximab IgG1 Gluc Exatecan (Molecule 2) and payload Exatecan (Molecule 4) treatment in NCI-H446 cells. One representative dose-response curve is shown as means from triplicate values±SD.

FIG. 16 In vitro dose-response curves for hu14.18-IgG1.4 (K322A)-delK-βGluc Exatecan (Molecule 1), control ADC Rituximab IgG1 βGluc Exatecan (Molecule 2) and payload Exatecan (Molecule 4) in MDA-MB-468 cells. One representative dose-response curve is shown as means from triplicate values±SD.

FIG. 17 In vitro dose-response curves for hu14.18-IgG1.4 (K322A)-delK-βGluc Exatecan (Molecule 1) and hu14.18-IgG1.4 (K322A)-delk (Molecule 3) in CHP-134 cells. Data are presented as means from triplicate values±SD. Curves depicted in figure are from two independent experiments.

Experiment 6: Safety Profile of ADC1: A Pilot i.v. Toxicity-TK Study in Cynomolgus Monkeys

To investigate the safety profile, ADC1 was administered by 30-min i.v. infusion to cynomolgus monkeys, three times with a week interval (on day 1, 8 and 15), at dosages of 4, 8, 16 and 32 mg/kg, and animals were sacrificed (on day 22) for gross and histopathological examination of a large panel of organs and tissues including peripheral nerve tissues. Also, clinical signs, bodyweight, clinical hematology and biochemistry parameters, as well as toxicokinetics (TK) were included in the study. ADC1 mainly induced dose-dependent effects in the hematolymphoid and gastro-intestinal system resembling Exatecan toxicity (De Jager 2000, Verschraegen 2000, Rowinsky 2005). Notably, no clinical signs of pain were observed during home cage observations or during handling of the monkeys for dose administration or when taking blood samples. No histopathological alterations were observed in examination of several peripheral nerve tissues (i.e. fibular, tibial, and sciatic nerves) or central brain tissues. Adequate exposure levels of plasma conjugated antibody was shown associated with very low plasma concentration of unconjugated (released) Exatecan. The absence of pain in this study is of eminent importance since pain or allodynia was the dose-limiting toxicity in the treatment of oncology patients with approved GD2 (IgG1) antibodies such as dinutuximab (ch14.18/SP2/0, Unituxin), dinutuximab beta (ch14.18/CHO or APN311) and naxitamab (hu3F8). Dinutaximab beta as tested in cynomolgus monkeys showed clinical signs that were interpreted as acute pain as confirmed by the observed histopathology changes in the peripheral nervous system or innervation structures of non-neuronal tissues (EMA/263814/2017 Assessment report dinutuximab beta Apeiron). The labelling of dinutuximab beta as well as of the original dinutuximab, and naxitamab antibodies confirm safety information on acute pain (treatment) issues. Even with GD2 antibodies mutated only for CDC effector functionality by K322A mutation, only partly relieved pain (Dobrenkov and Cheung 2014; Navid et al 2014) and opioid use was still required (Harman et al 2019).

Overall, the monkey safety data and mouse oncology model data with the novel ADC show tumors are killed by targeted Exatecan-mediated apoptosis (not by CDC and/or ADCC mechanisms) without the pain associated with naked anti-GD2 antibody administration alone.

### Experiment 7: Evaluation of a Peripheral Nervous System Sparing ADC (Molecule 1) in Rats and Cynomolgus Monkeys

To study the initial safety profile of Molecule 1 in monkeys and rats both expressing the identical GD2 glycotope, which can be targeted by the hu14. 18 Ab variant (FDA BLA #125516, 2014; EMA/263814/2017, 2017) used for conjugation, Molecule 1 showed the expected Exatecan toxicity profile but did not cause any PNS injury after weekly i.v. repeat-dosing in monkeys and rats. In addition, no indication of pain signals was observed in the normal behavior of animals in their home-cages such as locomotor activity, rearing, and exploration, or when handled.

### Methods

The general toxicology program comprised of pilot repeat-dose toxicity studies with Molecule 1 administered once weekly by intravenous infusion for 3 consecutive times (day 1, 8 and 15) in Wistar rats and cynomolgus monkeys, including toxicokinetic evaluations for exposure confirmation of the ADC (conjugated payload analyzed by Liquid Chromatography-Tandem Mass Spectrometry (LC-MS/MS) and total antibody analyzed by immunoassay) and released payload in plasma (analysed by Ultra-Performance LC-MS/MS). Necropsy for macro- and microscopic examinations was scheduled on day 22.

Intravenous dose formulations: Molecule 1 (Ab manufactured by EMD Serono, Billerica, USA, and conjugated by Sterling Deeside Ltd., Flintshire, United Kingdom) was formulated in a vehicle solution (control groups) containing 10 mM histidine, 40 mM NaCl, 6% trehalose dihydrate, 0.05% polysorbate 20 (pH 5.5) and stored deeply frozen (−60° C.) until use. The stock concentration (10 mg/mL) and dilutions of Molecule 1 were verified for stability and concentration prior to dosing (Merck KGaA, Darmstadt, Germany). The test item or vehicle was administered by intravenous (iv) slow bolus infusion to rats (tail vein) and 30-minute infusion to cynomolgus monkeys (peripheral vein). General parameters for the evaluation of toxicity. Toxicity indices consisted of daily clinical observations, body weight, food consumption, clinical pathology, immunophenotyping, gross pathology, organ weights, and histopathology. Necropsies included examination of the carcass; external body orifices; abdominal, thoracic, and cranial cavities; and organs. Tissues collected at necropsy were preserved in 10% neutral-buffered formalin, Davidson's (eye and optic nerve) or modified Davidson's (testis) fixative and were processed for routine histologic examination (paraffin wax-embedded tissues and HE stain). Selected organs were weighed prior to fixation. Additionally, unscheduled necropsy and histology was performed on moribund animals.

### B. Methodology

### 3-Week Pilot Rat Toxicity and Toxicokinetic Study

Molecule 1 was administered weekly by intravenous infusion for 3 consecutive weeks to four groups of CrI:WI (Han) rats (5 rats/sex/group; Charles River Laboratories, Sulzfeld, Germany) at doses of 0 (vehicle control), 10, 30, and 60 mg/kg of Molecule 1. Satellite groups of 3 rats/sex/group were used for toxicokinetic evaluation of three analytes. Methods: Rats were housed in groups of 2-3 per cage. Physical appearance, behavior and clinical signs of the rats were observed daily. Body weight was recorded prior to the start of treatment and thereafter daily up to the end of the study. Food consumption was recorded for each cage at weekly intervals up to the end of the study by weighing the food per cage which had not been consumed. Hematological examination and clinical chemistry tests were performed on 5 rats/sex/group, one week after the final dosing period (day 22). Hematological parameters were analyzed with the ADVIA2120i auto-analyzer and clinical chemistry parameters with ADVIA 1800 auto-analyzer (both Siemens Healthcare Diagnostics, GmbH). Blood samples (0.15 mL in lithium heparin tubes) from satellite and main groups (scarce sampling) were taken for bioanalysis and toxicokinetic (TK) evaluation from all animals on Days 1 and 15 up to one week thereafter at the following time-points: 0 (pre-dose), and 0.5, 4, 24, 48, 96 (only Day 1), and 168 h post-dose (before next dose). As control, vehicle treated animals were sampled on days 1 and 15 (at 4 h after first and last dosing).

### ii. 3-Week Pilot Monkey Toxicity and Toxicokinetic Study Including Functional Assessment of Cardiovascular (CV) Parameters and Respiratory Rate

Purpose-bred, naïve cynomolgus monkeys (Macaca fascicularis) were purchased from Envigo (Venray, The Netherlands). Monkeys were born and bred in Vietnam and quarantined in Camarles (Camarney SLU, Spain) before transport to the test facility in Ivrea (Italy). Monkeys were housed in an air-conditioned room (22±2° C.), with 15-20 air changes per hour, a relative humidity of 55±15% and artificial lighting with a circadian cycle of 12 hours from 7 a.m. to 7 p.m. Animals were housed in groups in monkey enclosures fixed to the floor. The front wall and top of the enclosures are made of stainless-steel bars, while the lateral and back walls consist of colored plastic material. In this study, the experimental groups consisted of one animal per sex per group, but extra non-dosed animals were present to ensure social housing groups of two or three subjects. Non-human primate housing meets the Italian requirements for laboratory animal welfare including an environmental enrichment program. At the start of treatment, male and female animals were approximately 3-4 years old with body weight ranging between 3.1-3.8 kg. Molecule 1 was administered weekly by 30-minutes iv infusion to 1 male and 1 female cynomolgus monkey per group at three dose levels (4, 8 and 16 mg/kg) of test item for 3 consecutive weeks. One additional male monkey was infused at a fourth higher dose level (32 mg/kg) to determine the maximum tolerability.

Mortality and clinical signs (physical appearance, behavior, and general signs) were recorded twice daily as well as at the end of infusion (until 30-min post-dose), and body weight was recorded once weekly from one week before commencement of treatment throughout the study period. Food and water intake was conducted daily (per cage). Hematology (ADVIA2120i Siemens analyzer) were investigated in all monkeys during the pre-treatment period, and on day 3 (i.e., 48 h post-1st dose), day 8 (168 h before 2nd dose), day 15 (before 3rd dose), day 17 (i.e., 48 h post-3rd dose), and day 22 (before sacrifice). Immunophenotyping (FACLyric Becton Dickinson flow cytometer, Becton Dickinson antibodies) was performed on the same days to measure absolute and relative counts of total B cells (CD3−, CD20+), total (CD3+), helper (CD3+, CD4+) and cytotoxic T cells (CD3+, CD8+) and natural killer cells (CD3−, CD16+). Clinical chemistry (AU480 Beckman Coulter analyzer) was performed during the pre-dose period, and on day 15 and day 22.

Blood samples (0.6 mL in lithium heparin tubes) for bioanalysis and TK evaluation were taken from each animal on Days 1 and 15 up to one week thereafter at the following time-points: 0 (pre-dose), and 0.5 (=end of infusion), 2, 6, 24, 48, 72, 120 and 168 h post-dose (before next dose).

iii. Integrated Electrocardiogram and Arterial Blood Pressure Assessments

As an integral part of the pilot monkey DRF toxicity study, heart rate (HR), electrocardiogram (ECG), and arterial blood pressure (systolic and diastolic BP), were measured in all monkeys at baseline (pre-dosing) and within 30-minutes at the end of infusion of the last dose (day 15). Animals were trained to take recordings under conscious, temporarily restrained condition while sitting in a chair. Three consecutive measurements of systolic and diastolic arterial blood pressures were performed before respiratory rate and the ECG measurement session. Evaluations were performed on at least 10 representative ECG complexes. Respiratory rate was measured immediately after arterial blood pressure measurements by counting the respiratory movements over one minute. Rectal temperature was recorded in all animals, twice pre-dosing, and on days 1, 8 and 15, at the end of infusion (within 30 minutes post-dose) and 24 hours (+30 minutes) post-dose.

Methods: ECG electrodes (single use foam electrodes) were placed on each animal according to Standard II Leads. The signals were digitized (A/D converter ACQ-7700, DSI, St. Paul, Minnesota, USA) and continuously recorded once the heart rate reached a steady state, using a software package (Ponemah Physiology Platform 5.20, from DSI supplier). A cuff for arterial blood pressure (BP) measurement was connected to a blood pressure monitor (Monitoring vital signs unit CARESCAPE™ V100, GE Healthcare, Milwaukee, Wisconsin, USA) and placed on one of the forearms. No statistical analyses were performed due to the low number of animals per group.

### Experimental Findings:

### Pilot 3-Week Toxicology Study in Wistar Rats

Following three consecutive, weekly i.v. injection at three dose levels of Molecule 1 resulted in premature sacrifice of 3 out of 16 rats dosed at 60 mg/kg due to clinical signs (sunken flank, coat piloerection, ptosis) and body weight decline of −25% in males and −12% in females at the end of the study (day 21) compared to control (FIG. 13). After each weekly dosing, there were transient BW losses at 60 mg/kg with nadir values 3 to 4 days post-injection of up to −7% in males (up to −27% vs controls) and −10% in females (up to −15% vs controls) as compared to their weekly pre-dose BW values. At 30 mg/kg, body weight was reduced by −10% to −11% in both sexes at day 21 (compared to control). Over the 21-day observation period, body weight gain was almost absent at 60 mg/kg and almost 50% reduced at 30 mg/kg in both sexes, as compared to controls. Food consumption was dose-dependently reduced by 33-37% at 60 mg/kg and 16-19% at 30 mg/kg.

On day 22, one week after the last dose (day 15), clinical pathology measurements showed up to moderate decreases of erythrocytes, hemoglobin, and hematocrit (all parameters up to −29% at 60 mg/kg and up to −9% at 30 mg/kg) with reticulocyte increases (up to 90%) at the mid-dose indicative of regeneration, and without reticulocyte changes at the high dose. At 60 mg/kg, leucocytes, lymphocytes, and eosinophils appeared declined as well as partly at 30 mg/kg (eosinophils in males and lymphocytes in females). At the mid- and high dose, platelets were increased (up to 60%) whereas neutrophil counts showed a trend towards reduction (not statistically significant). The selected day 22 for hematological analysis may not have been optimal regarding the regenerative capacity of hematopoiesis in the rat. Hematotoxic effects described for Exatecan in rodents such as decreased neutrophils, lymphocytes, and thrombocytes (Verschraegen et al., 2000) may have been partly recovered 7 days after the final dose of Molecule 1. Regarding clinical blood chemistry, total protein (−14%) and albumin (−12%) were decreased at 60 mg/kg only. The latter effects were most probably related to a general catabolic metabolism in absence of any body weight growth over 3 weeks.

Microscopic examination of organs collected on day 22 revealed adverse findings in the hematolymphoid system (decreased cellularity and/or increased single cell necrosis in thymus, bone marrow (BM), and periarteriolar lymphoid sheath (PALS) of the spleen, and lymph nodes), gastrointestinal tract (increased single cell necrosis in the crypt regions), and reproductive organs (oocyte, granulosa cell, and seminiferous tubule degeneration), which reflect the pattern of an antimitotic cytotoxic agent. In the thymus, a moderate to marked loss of corticomedullary distinction was observed in the most severely affected rats. In addition, secondary findings consisting of reduced primary trabeculae, woven bone formation or fibrosis in the bone/bone marrow and skin ulcerations occurred in single rats especially in the 60 mg/kg dosed group. Woven bone formation and fibrosis can be regarded as an attempt to compensate for the disturbed bone formation. In four male rats of the 60 mg/kg group, skin wounds with ulcerations occurred probably due to an opportunistic infection (presence of coccoid bacterial colonies in microscopy slides) of commonly occurring skin changes (scratch or bite wounds) under circumstances of immunosuppression. The loss of germinal centers in lymph nodes of most rats noted from the low dose of 10 mg/kg and higher, indicated the high susceptibility of this specific B-cell compartment for the antimitotic activity of Molecule 1 in the rat Other observations at 60 mg/kg were the minimal extramedullary hematopoiesis in the liver and adrenals which are regarded as an adaptive phenomenon due to bone marrow suppression. The prematurely sacrificed female from the main 60 mg/kg group (day 14 after 2-times dosing) showed multifocal, moderate erosions in the cecum likely contributing to its poor clinical condition. Other findings in this rat were comparable to the rest in this group. The two prematurely sacrificed rats (female at day 5, male at day 18) of the satellite TK group were not examined for histopathology.

Microscopic evaluation of the rat dorsal root ganglia (cervical, thoracal, lumbar) and peripheral nerves including optic, tibial, fibular, and sciatic nerves did not show any abnormalities.

### Toxicokinetic Evaluation

Overall plasma exposure increased proportionally to the increasing dose over the tested dose range for total antibody, conjugated (Exatecan), and unconjugated (released Exatecan) with accumulation of the ADC at 10 and 30 mg/kg treated groups but not at the 60 mg/kg group. The maximum plasma concentration of very low plasma levels of unconjugated Exatecan was observed between 0.5 to 4 h with similar half-life as the ADC (about 2-3 days) indicating a formation rate-limiting process from the ADC. No relevant gender differences in plasma exposure were apparent.

vi. Pilot 3-Week Toxicology Study with CV-Function Assessment in Cynomolgus Monkeys

Weekly i.v. dosing for 3 consecutive times at three dose levels of Molecule 1 to cynomolgus monkeys was tolerated. A fourth higher dose of 32 mg/kg Molecule 1 infused to a single male monkey was not tolerated after two times dosing (day 1 and day 8) and required sacrifice at day 14. Therefore, the dose of 32 mg/kg was considered to exceed the MTD in this study.

Up to the MTD, monkeys showed dose-dependent gastrointestinal clinical signs (i.e. episodes of soft feces and/or diarrhea) associated with body weight decrease. At the MTD of 16 mg/kg slight episodes of soft feces and/or diarrhea were noted in both animals and in the female at 8 mg/kg. This was associated with gradual BW reduction over time of −11% (day 14) to −16% (day 21) in the female at 16 mg/kg, but not in the male. Mild BW decreases were noted at 8 mg/kg in both sexes and at 4 mg/kg in the male monkey. Hematology investigations in monkeys over time (FIG. 14) showed minimal to slight decreases in red blood cells, hemoglobin, and hematocrit (up to −27% vs. baseline values, in both sexes at 16 mg/kg) at all dose levels, with a limited dose-relationship. A regenerative response (i.e., up to 5-fold increase in reticulocyte counts) was seen at 4 and 8 mg/kg, while moderate to severe decreases (up to about-95%, on day 8) were noted at 16 mg/kg for the same parameter. Neutrophil counts were transiently reduced (<2.0 10³/μL) in the male monkey on day 8 and 15 at 16 mg/kg but still above the lower limit of normal reference values (=1.10×10³/μL, mean 5.77±3.57×10³/μL as reported in Park et al., 2016).

At 16 mg/kg histopathology was limited to changes in the lymphoid system (hypocellularity in lymph nodes and thymus). The mild effects in the thymus in all monkeys treated at 8 and 4 mg/kg might be confounded by secondary stress-related effects. No histopathological changes were noted in the GI-tract.

Microscopic evaluation of the peripheral nerves including optic, tibial, fibular, and sciatic nerves did not show any test item-related findings at any dose tested including in the male monkey exceeding the MTD (day 14 necropsy).

### Cardiovascular Functions

No relevant changes were measured up to the MTD regarding heart rate (HR), ECG (QT/HR-corrected QT), systolic, diastolic, and mean blood pressure, nor for respiratory rate and body temperature.

### Toxicokinetic Evaluation

Overall plasma exposure increased proportionally to the increasing dose over the tested dose range for total antibody, conjugated (Exatecan), and unconjugated (released Exatecan) without accumulation of any analyte. Only in a single monkey (male) a drop in exposure was observed after repeated administration of 4 mg/kg, indicating potential ADA formation in this subject. The maximum plasma concentration of very low levels of unconjugated Exatecan was usually observed after 6 h with similar half-life as the ADC (about 2-3 days) indicating a formation rate-limiting process from the ADC. No relevant gender differences in plasma exposure were apparent.

### Discussion of Experimental Findings

Despite improvement of survival rate, acute neuropathic pain is a common and key dose-limiting adverse event (AEs) associated with peripheral neuropathy observed with GD2 immunotherapy of currently marketed anti-GD2 antibodies (dinutuximab, naxitamab) in the treatment of patients suffering from neuroblastoma and osteosarcoma. In pilot toxicity/TK studies with rats and cynomolgus monkeys with weekly iv dosing (3-times) of the Exatecan-based ADC Molecule 1, no injury of the PNS was observed on day 22 and no indication of pain was noted in their behavior. Molecule 1 led to potent anti-tumor activity in xenografted mice models expressing GD2, even after single dose application. Together these nonclinical data show a positive benefit-risk ratio likely to improve the clinical perspective of treatment of GD2-expressing tumors in children and adult patients against current GD2 immunotherapy. In general, no unusual signs of defensive behavior such as hyper-responsiveness, anxiety, or agitation were noted during handling that might have indicated mechanical allodynia. Also, in their home-cage behavior no signs indicative of pain were observed such as reduced locomotor activity, rearing, and exploration. In addition, trained monkeys sitting in a chair received 30-minute i.v. infusion without issues and subsequent measurement of CV function after 1 h that may respond to pain stimuli such as tachycardia or increased blood pressure, albeit non-specifically, appeared to remain normal in monkeys.

In contrast, in a toxicity study with cynomolgus monkeys infused 4 h/day for 10 consecutive days (30 and 100 mg/m²/day) with dinutuximab beta histopathological changes were noted on day 15 in the peripheral nerves and visceral nervous innervation of organs (EMA/263814/2017, 2017). These observations together with clinical signs in these monkeys (e.g. hypoactivity) was interpreted as signs most probably relating to acute pain induction due to the test item targeting GD2 within the peripheral nervous system. After treatment of melanomas with anti-GD2 mAb (14G2a; ch14.18 is derived from this murine IgG2a isotype), some patients developed sensorimotor demyelinating polyneuropathy (Yuki et al., 1997). No toxicology studies have been conducted with monkeys or other non-rodents with naxitamab (FDA BLA #761171, 2020) or with dinutuximab (Unituxin®, FDA BLA #125516, 2014).

In DRF toxicity studies, no microscopic changes were noted in several examined peripheral nerves of monkeys and rats after repeated Molecule 1 application (sciatic, tibialis, fibularis, and optical nerves, and DRG at different levels in rats) while adequate exposure in plasma to the ADC was confirmed. Clinical signs in monkeys were limited to occasional incidences of mild soft feces and diarrhea at the MTD of 16 mg/kg only as well as BW reduction in the female (−16%) but not the male. Dose-dependent BW gain reduction and slight food intake decrease was observed in rats mainly. Due to the poor condition of 3 out of 16 rats at 60 mg/kg without BW gain, the high dose in this DRF rat study was considered to exceed the MTD. From clinical and histopathological examination, monkeys and rats showed hematolymphoid and GI-tract effects resembling Exatecan toxicity (Verschraegen et al., 2000, Rowinsky 2005) attributed to the relatively low plasma levels of unconjugated Exatecan released over a week from the ADC. Neutropenia is regarded as the main dose-limiting toxicity with Exatecan mesylate as observed in oncology patients and animals together with other hematotoxic effects (such as anemia, lymphocytopenia, and thrombocytopenia) and GI-tract effects.

As detailed in Experiment 7, no signs of immunogenicity were observed in rats and monkeys following repeat-dosing of Molecule 1 over three weeks (except for one low dosed monkey) as judged by the TK profiles showing normal dose-proportionality and regular TK curves, and absence of unusual toxicities. The absence of immunogenicity is likely explained by the induced strong immunosuppressive effects of Molecule 1 (e.g. absence of germinal centers, a main B-cell compartment). Other non-hematotoxic effects observed with Molecule 1, such as anti-proliferative changes in reproduction organs/tissues in female and male rats, can be attributed to the antimitotic effects of Exatecan (Verschraegen et al., 2000, Rowinsky 2005).

Changing the tumor killing concept by replacing it with targeted delivery of a non-neurotoxic payload, and eliminating any Ab Fc-effector functionality, prevented PNS injury in two tested animal species. The high advantage of this consistent outcome is that Molecule 1 may at least omit or reduce potential harm to the PNS of patients.

In some embodiments of the present invention, therapeutic methods describe the PNS sparing activity of Molecule 1 so that neurological disorders of the eye (e.g., mydriasis, blurred vision, unequal pupils, or photophobia) may be prevented.

1. An antibody drug conjugate (“ADC”) comprising a growth inhibitory agent and/or an anti-proliferative agent linked to an antibody by way of a linker, wherein the antibody comprises the anti-GD2 antibody light chain variable region of SEQ ID NO: 1, the anti-GD2 antibody heavy chain variable region of SEQ ID NO:2, and an Fc region with one mutation decreasing complement fixation relative to antibody-dependent, cell-mediated cytotoxicity, wherein the one mutation causes an absolute decrease in complement fixation.

2. The antibody of claim 1, wherein the one mutation eliminates complement fixation.

3. The antibody of claim 1, wherein the Fc region is derived from IgG.

4. The antibody of claim 3, wherein the IgG is IgG1.

5. The antibody of claim 1, further comprising a CH1 domain.

6. The antibody of claim 5, wherein the Fc region is derived from IgG.

7. The antibody of claim 5, wherein the IgG is IgG1.

8. The antibody of claim 1, further comprising a CL domain.

9. The antibody of claim 8, further comprising a CH1 domain.

10. The linker of claim 1, wherein said linker is cleavable by glucuronidase.

11. The linker of claim 1, wherein said linker is cleavable by legumain.

12. The growth inhibitory agent of claim 1, wherein said growth inhibitory agent is Exatecan.

13. The ADC of claim 1 which has an average drug-to-antibody ratio in the range of 1-10.

14. The ADC of claim 1 which has an average drug-to-antibody ratio in the range of 2-4.

15. The ADC of claim 1, wherein the antibody or antigen binding fragment thereof comprises a heavy chain variable region comprising an amino acid sequence at least 85%, 86%, 87%, 88%, 89%, 90%, 91%, 92%, 93%, 94%, 95%, 96%, 97%, 98%, or 99%-homologous to the heavy chain variable region amino acid sequence set forth in SEQ ID NO: 2 and a light chain variable region comprising an amino acid sequence at least 85%, 86%, 87%, 88%, 89%, 90%, 91%, 92%, 93%, 94%, 95%, 96%, 97%, 98%, or 99% homologous to the light chain variable region amino acid sequence set forth in SEQ ID NO:1.

16. A pharmaceutical composition comprising the ADC of claim 1 and a pharmaceutically acceptable excipient.

17. A method of treating cancer in a subject, comprising administering to the subject a therapeutically effective amount of the pharmaceutical composition of claim 16.

18. An antibody drug conjugate (“ADC”) comprising a growth inhibitory agent and/or an anti-proliferative agent linked to an anti-GD2 antibody by way of a linker, wherein said anti-GD2 antibody comprises the amino acid sequence of SEQ ID NO:4 and the amino acid sequence of SEQ ID NO:1, said linker is β-glucuronide, and said growth inhibitory agent is Exatecan.

19. The ADC of claim 18, wherein the antibody or antigen binding fragment thereof comprises a heavy chain variable region comprising an amino acid sequence at least 85%, 86%, 87%, 88%, 89%, 90%, 91%, 92%, 93%, 94%, 95%, 96%, 97%, 98%, or 99% homologous to the heavy chain variable region amino acid sequence set forth in SEQ ID NO: 4 and a light chain variable region comprising an amino acid sequence at least 85%, 86%, 87%, 88%, 89%, 90%, 91%, 92%, 93%, 94%, 95%, 96%, 97%, 98%, or 99% homologous to the light chain variable region amino acid sequence set forth in SEQ ID NO:1.

20. A pharmaceutical composition comprising the ADC of claim 18 and a pharmaceutically acceptable excipient.

21. A method of treating cancer in a subject, comprising administering to the subject a therapeutically effective amount of the pharmaceutical composition of claim 20.

22. A method, as claimed in claim 21, wherein the administration of said pharmaceutical composition is associated with a decrease in peripheral neuropathy associated with the administration of other ADCs approved for human use.
